(19) United States
(12) Patent Application Publication
(10) Pub. No.: US 2026/0306479 A1
(43) Pub. Date: Oct. 8, 2026

(54) SUBSTITUTED NUCLEOSIDE ANALOGS AS PRMT5 INHIBITORS

(71) Applicant: Lupin Limited, Maharastra Mumbai (IN)

(72) Inventors: Prathap Sreedharan NAIR, Maharashtra Pune (IN); Ganesh Bhausaheb GUDADE, Maharashtra Pune (IN); Shankar Bhaskar BHAGWAT, Maharashtra Pune (IN); Amol Maruti YADAV, Maharashtra Pune (IN); Chaitanya Prabhakar KULKARNI, Maharashtra Pune (IN); Milind Dattatraya SINDKHEDKAR, Maharashtra Pune (IN); Venkata P. PALLE, Maharashtra Pune (IN); Rajender Kumar KAMBOJ, Maharashtra Pune (IN)

(73) Assignee: Lupin Limited, Maharastra Mumbai (IN)

(21) Appl. No.: 19/705,153

(22) Filed: Jun. 11, 2026

Related U.S. Application Data
(62) Division of application No. 17/782,214, filed on Dec. 2, 2020, now Pat. No. 12,673,054.

(30) Foreign Application Priority Data
Dec. 3, 2019 (IN) 201921049775

(51) Int. Cl. A61K 31/473; A61K 31/437; A61K 31/4375; A61K 31/536; A61P 35/00

(52) U.S. Cl. CPC A61K 31/473; A61K 31/437; A61K 31/4375; A61K 31/536; A61P 35/00

(57) ABSTRACT

The invention relates to substituted nucleoside analogues of formula (1), pharmaceutically acceptable salts thereof and pharmaceutical compositions for treating diseases, disorders or conditions associated with the overexpression of PRMT5 enzyme. The invention also relates to methods of treating diseases, disorders or conditions associated with the overexpression of PRMT5 enzyme.

### CROSS-REFERENCE TO RELATED APPLICATIONS

This Application is a divisional of U.S. application Ser. No. 17/782,214, filed Jun. 3, 2022, which is a national stage entry of International Application No. PCT/IB2020/061372, filed Dec. 2, 2020, which claims priority to IN Application No. 201921049775, filed Dec. 3, 2019, the contents of which are hereby incorporated by reference in their entirety.

### FIELD OF THE INVENTION

The invention relates to substituted nucleoside analogues of formula (I), pharmaceutically acceptable salts thereof and pharmaceutical compositions for treating diseases, disorders or conditions associated with the overexpression of PRMT5 enzyme. The invention also relates to methods of treating diseases, disorders or conditions associated with the overexpression of PRMT5 enzyme.

### BACKGROUND OF THE INVENTION

Methylation of proteins is a common post-translational modification that affects the protein's activity and its interaction with other biological molecules. N-methylation typically occurs on the nitrogen atoms of arginine, lysine and histidine residues and there are different families of enzymes that catalyze the methylation reaction, each being specific to the amino acid residue that will be methylated.

A family of 9 enzymes, called Protein Arginine N-Methyl Transferases (PRMTs), are responsible for the methylation of the guanidinium group of arginine. The guanidinium group of arginine bears 2 terminal nitrogen atoms that undergo monomethylation or dimethylation. Depending on the type of dimethylation, the enzymes are further classified as type I or type II. Type I PRMTs catalyse the monomethylation or the asymmetric dimethylation whereas type II enzymes catalyse the symmetric dimethylation. Some of the substrates that undergo methylation are histones, Sm ribonucleoproteins, MRE11 and p53 binding protein 1.

The methylation of arginine side-chains has an important role to play in various cell functions that include transcription activation as well as transcription repression, mRNA translation, pre-mRNA splicing, protein trafficking and signal transduction. It also occurs on myriad substrates. The enzymatic activity of the PRMTs hence affects cellular processes like cell proliferation, repair of damaged DNA as well as cell cycle and cell death. It has been shown that PRMT enzyme-mediated hypermethylation leads to certain disease conditions like cancer (Nature Reviews Cancer 2013, 13, p37; Cellular and Molecular Life Sciences 2015, 72, p2041; Trends in Biochemical Sciences 2011, 36, p633).

At present, the most studied type II enzyme is PRMT5, which is conserved across the eukaryotic organisms. Overexpression of PRMT5 is linked with carcinogenesis and decreased patient survival in several human malignancies (Cell Mol Life Sci., 2015, 72, p2041). PRMT5 directly interacts with proteins often dysregulated or mutated in cancers, hence a putative oncogene (Mol Cell Biol, 2008, 28, p6262). PRMT5 mediated transcriptional repression of tumor suppressor genes like p53, RB-1, ST7, or upregulation of Cyclin D1, CDK4, CDK6, eLF4E, MITF, FGFR3 associate with the oncogenesis in both solid tumors and hemaological malignancies. PRMT5 is located in the nucleus as well as the cytoplasm and its overexpression has been linked to a wide range of cancers including, but not limited to, glioblastoma multiforme (Oncogene, 2017, 36, p263), prostate cancer (Oncogene, 2017, 36, p1223), and pancreatic cancer (Science, 2016, 351, p1214), mantle cell lymphoma (Nature Chemical Biology, 2015, 11, p432), non-Hodgkin's lymphomas and diffuse large B-cell lymphoma (Journal of Biological Chemistry, 2013, 288, p35534), acute myeloid leukemia (Leukemia, 2018, 32, p499), acute lymphoblastic leukemia (AACR; Cancer Research 2017;77(13 Suppl):Abstract nr 1128), multiple myeloma (Leukemia, 2018, 32, p996), non-small cell lung cancer (The Biochemical Journal, 2012, 446, p235), small cell lung cancer (AACR; Cancer Research 2017;77(13 Suppl):Abstract nr DDT02-04), breast cancer (Cell Reports, 2017, 21, p3498), triple negative breast cancer (AACR; Cancer Res 2015;75(15 Suppl):Abstract nr 4786), gastric cancer (International Journal of Oncology, 2016, 49, p1195), colorectal cancer (Oncotarget, 2015, 6, p22799), ovarian cancer (J Histochem Cytochem 2013, 61, p206), bladder cancer (Clinical Cancer Research, 2018, CCR-18-1270), hepatocellular cancer (Oncology Reports, 2018, 40, p536), melanoma (PLoS One, 2013, 8, e74710; J Clin Invest. 2018, 128, p517), sarcoma (Oncology Letters, 2018, 16, p2161), oropharyngeal squamous cell carcinoma (Oncotarget, 2017, 8, p14847), chronic myelogenous leukemia (J Clin Invest, 2016, 126, p3961), epidermal squamous cell carcinoma (Carcinogenesis, 2017, 38, p827), nasopharyngeal carcinoma (Oncology Reports, 2016, 35, p1703), neuroblastoma (Molecular Oncology, 2015, 9, p617), endometrial carcinoma (Gynecol Oncol., 2016, 140, p145), cervical cancer (Pharmazie, 2018, 73, p269). These findings have led to further research which show that inhibiting PRMT5 reduces cell proliferation (Molecular and Cellular Biology 2008, 28, p6262, The Journal of Biological Chemistry 2013, 288, p35534).

Inhibitors of arginine methyl transferases were first disclosed in 2004 by Cheng et al in the Journal of Biological Chemistry—Vol. 279 (23), p.23892. Since then, various other compounds and substances having greater selectivity towards either type I or type II arginine methyl transferases have been disclosed. Other publications that disclose small molecules as inhibitors in relation to PRMT5 are: WO2011077133, WO2011079236, WO2014100695, WO2014100716, WO2014100719, WO2014100730, WO2014100734, WO2014128465, WO2014145214, WO2015200677, WO2015200680, WO2015198229, WO2016022605, WO2016034671, WO2016034673, WO2016034675, WO2016038550, WO2016135582, WO2016145150, WO2016178870, WO2017032840, WO2018160824, WO2018152501, WO2018085818, WO2018065365, WO2019116302A1, WO2020033288A1, WO2020205867A1 and ACS Medicinal Chemistry Letters 2015, 6, p408.

### SUMMARY OF THE INVENTION

In accordance with one aspect, the invention provides compound of general formula (I), a stereoisomer thereof, a tautomeric form thereof, or a pharmaceutically acceptable salt thereof,

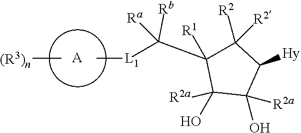

wherein,
L₁ is selected from —CR^{a}R^{b}—, —NR^{a}—, S, and O;
R^{a} and R^{b} are independently selected at each occurrence from hydrogen, substituted or unsubstituted alkyl, and substituted or unsubstituted cycloalkyl;
ring A is selected from formula (i) to (viii), wherein the substituent R³ on ring A may be substituted on any of the ring C atoms,

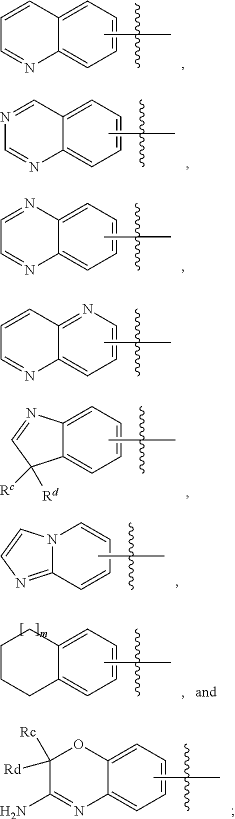

R^{c} and R^{N} are substituted or unsubstituted alkyl or together with the carbon atoms to which R^{c} and R^{d1} are attached form a C₃-C₆ cycloalkyl ring;
Hy is selected from formula (a-1) to (d-1),

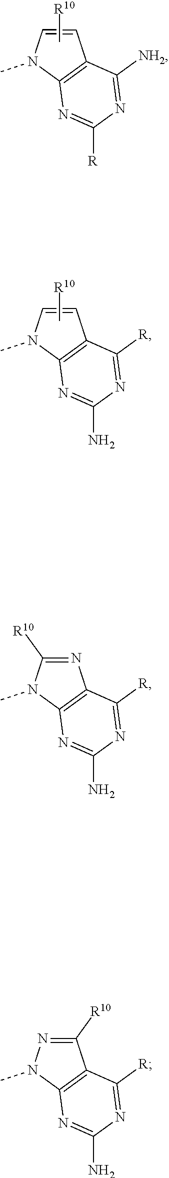

R is selected from —NR⁴R⁵, substituted or unsubstituted alkyl and cycloalkyl;
R¹ and R² together with the carbon atoms to which they are attached form a bond in order to form a —C═C—; or R¹ and R² together with the carbon atoms to which they are attached form a cyclopropane ring;
R^{2′} and R^{2a} which may be same or different and are independently selected at each occurrence from hydrogen and substituted or unsubstituted alkyl;
R³ is independently selected at each occurrence from halogen, cyano, nitro, substituted or unsubstituted alkyl, —OR⁶, —NR⁷R⁸, substituted or unsubstituted cycloalkyl, —C(O)OH, —C(O)O-alkyl, —C(O)R⁹, —C(O)NR⁷R⁸, —NR⁷C(O)R⁹, substituted or unsubstituted aryl, substituted or unsubstituted heteroaryl, substituted or unsubstituted heterocyclyl and haloalkyl;
R⁴ and R⁵ are independently selected from hydrogen, substituted or unsubstituted alkyl, and substituted or unsubstituted cycloalkyl;
R⁶ is selected from hydrogen, substituted or unsubstituted alkyl, and substituted or unsubstituted cycloalkyl;
R⁷ and R⁸ are independently selected from hydrogen, substituted or unsubstituted alkyl, and substituted or unsubstituted cycloalkyl;
R⁹ is selected from substituted or unsubstituted alkyl and substituted or unsubstituted cycloalkyl;
R¹⁰ is selected from hydrogen, halogen, and substituted or unsubstituted alkyl;
‘m’ is an integer ranging from 0 to 1, both inclusive;
‘n’ is an integer ranging from 0 to 4, both inclusive;
when an alkyl group is substituted, it is substituted with 1 to 4 substituents independently selected from oxo (═O), halogen, cyano, cycloalkyl, aryl, heteroaryl, heterocyclyl, —OR^{7a}, —C(═O)OH, —C(═O)O(alkyl), —NR^{8a}R^{8b}, —NR^{8a}C(═O)R^{9a}, and —C(═O)NR^{8a}R^{8b};
when the heteroaryl group is substituted, it is substituted with 1 to 4 substituents independently selected from halogen, cyano, alkyl, haloalkyl, perhaloalkyl, cycloalkyl, heterocyclyl, —OR^{7a}, —NR^{8a}R^{8b}, —NR^{7a}C(═O)R^{9a}, —C(═O)NR^{8a}R^{8b};
when the aryl group is substituted, it is substituted with 1 to 4 substituents independently selected from halogen, cyano, alkyl, haloalkyl, perhaloalkyl, cycloalkyl, heterocyclyl, —OR^{7a}, —NR^{8a}R^{8b}, —NR^{7a}C(═O)R^{9a}, —C(═O)NR^{8a}R^{8b};
when the cycloalkyl group is substituted, it is substituted with 1 to 4 substituents independently selected from halogen, cyano, alkyl, haloalkyl, perhaloalkyl, —OR^{7a}, —NR^{8a}R^{8b}, —NR^{7a}C(═O)R^{9a}, —C(═O)NR^{8a}R^{8b};
R^{7a} is selected from hydrogen, alkyl, perhaloalkyl, and cycloalkyl;
R^{8a} and R^{8b} are each independently selected from hydrogen, alkyl, and cycloalkyl; and
R^{9a} is selected from alkyl and cycloalkyl.

The details of one or more embodiments of the invention set forth in below are only illustrative in nature and not intended to limit to the scope of the invention. Other features, objects and advantages of the inventions will be apparent from the description and claims.

According to one embodiment, the invention provides compounds having the structure of formula (II), a stereoisomer thereof, or a pharmaceutically acceptable salt thereof,

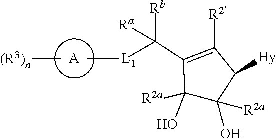

wherein,
Ring A, Hy, L₁, R^{2a}, R^{2′}, R^{a}, R^{b}, R³ and n are as defined herein above.

According to another embodiment, the invention provides compounds having the structure of formula (III), a stereoisomer thereof, or a pharmaceutically acceptable salt thereof,

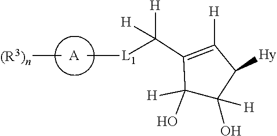

wherein, L₁ is —CR^{a}R^{b}— or O;
Ring A is

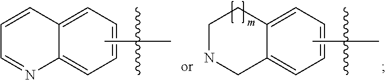

Hy is selected from formula (a-1), (b-1) and (d-1),

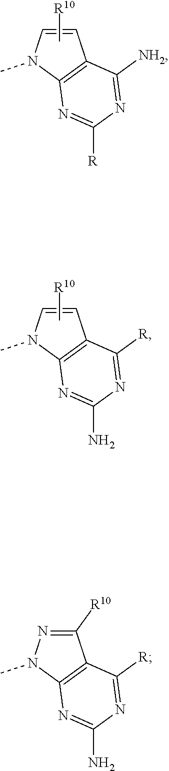

R is selected from —NR⁴R⁵, substituted or unsubstituted alkyl and cycloalkyl; R⁴ and R⁵ are independently selected from hydrogen; R^{2′} is hydrogen; R³ is independently selected at each occurrence from halogen, substituted or unsubstituted alkyl, —OR⁶ and —NR⁷R⁸; R⁷ and R⁸ are independently selected from hydrogen; R¹⁰ is hydrogen; ‘in’ is 1;
‘n’ is an integer ranging from 0 to 3, both inclusive.

According to one embodiment, there are provided compounds of formula (I), (II) and (III) wherein the compound is in the form of the free base or is a pharmaceutically acceptable salt thereof.

In any of the above embodiments, R is methyl.

According to another embodiment, there are provided compounds of formula (I), (II) and (III) wherein Hy is selected from,

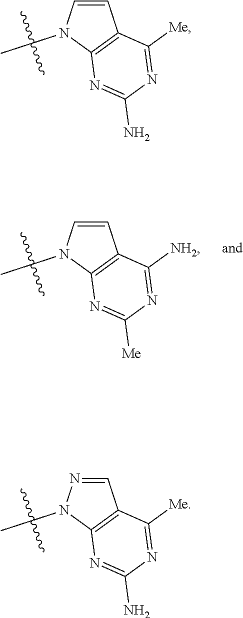 [SMILES: *n1ccc2c(C)nc(N)nc21 ; *n1ccc2c(N)nc(C)nc21 ; *n1ncc2c(C)nc(N)nc21]

In another aspect of the invention, there are provided compounds of formula (I), (II) and (III) or a pharmaceutically acceptable salt thereof for treating the diseases, disorders, syndromes or conditions associated with PRMT5 enzyme.

In one embodiment of the present invention, there are provided compounds of formula (I), (II) and (III) or a pharmaceutically acceptable salt thereof for treating diseases, disorders, syndromes or conditions by inhibition of PRMT5 enzyme.

In another aspect of the invention, there are provided compounds of formula (I), (II) and (III) or a pharmaceutically acceptable salt thereof for use as a medicament.

In another aspect of the invention, there are provided compounds of (I), (II) and (III) or a pharmaceutically acceptable salt thereof for use in treating the diseases, disorders, syndromes or conditions associated with PRMT5.

In one embodiment of the present invention, there are provided compounds of formula (I), (II) and (III) or a pharmaceutically acceptable salt thereof for use in treating diseases, disorders, syndromes or conditions by the inhibition of PRMT5.

In another aspect of the invention, there is provided a method of inhibiting PRMT5 by using a compound selected from formula (I), (II) and (III) or a pharmaceutically acceptable salt thereof.

In another aspect of the invention, there is provided a method of treating diseases, disorders or conditions associated with PRMT5 by using a compound selected from formula (I), (II) and (III).

In another aspect of the present invention, a method of treating diseases, disorders or conditions is selected from glioblastoma multiforme, prostate cancer, and pancreatic cancer, mantle cell lymphoma, non-Hodgkin's lymphomas and diffuse large B-cell lymphoma, acute myeloid leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, and cervical cancer by using a compound selected from formula (I), (II) and (III) is provided.

In another aspect of the invention, there is provided a use of a compound selected from formula (I), (II) and (III) or a pharmaceutically acceptable salt thereof, for the manufacture of a medicament for treating, the diseases, disorders or conditions associated with PRMT5.

In another aspect, the invention provides a pharmaceutical composition comprising at least one compound of formula (I), (II) and (III) or a pharmaceutically acceptable salt thereof and at least one pharmaceutically acceptable excipient.

In another aspect, the invention provides a pharmaceutical composition comprising a therapeutically effective amount of compound of formula (I), (II) and (III) or a pharmaceutically acceptable salt thereof, for use in treating, the diseases, disorders or conditions associated with PRMT5 by administering to the subject in need thereof.

In another aspect of the present invention, wherein the use of compounds of formula (I), (II) and (III) or a pharmaceutically acceptable salt thereof for the diseases, disorders, syndromes or conditions associated by inhibition of PRMT5 are selected from the group consisting of glioblastoma multiforme, prostate cancer, and pancreatic cancer, mantle cell lymphoma, non-Hodgkin's lymphomas and diffuse large B-cell lymphoma, acute myeloid leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, and cervical cancer.

In another aspect, the invention provides a pharmaceutical composition comprising a therapeutically effective amount of compound of formula (I), (II) and (III) or a pharmaceutically acceptable salt thereof, for treating the diseases, disorders or conditions associated with PRMT5 by administering to the subject in need thereof.

In another embodiment of the invention the compounds, their stereoisomers or pharmaceutically acceptable salts thereof are:
(1S,2R,5R)-3-(2-(2-Amino-3-chloro-5-fluoroquinolin-7-yl)ethyl)-5-(2-amino-4-methyl-7H-pyrrolo[2,3-d]pyrimidin-7-yl)cyclopent-3-ene-1,2-diol;
(1S,2R,5R)-3-(((2-Amino-3-chloro-5-fluoroquinolin-7-yl)oxy)methyl)-5-(2-amino-4-methyl-7H-pyrrolo[2,3-d]pyrimidin-7-yl)cyclopent-3-ene-1,2-diol;
(1S,2R,5R)-5-(4-Amino-2-methyl-7H-pyrrolo[2,3-d]pyrimidin-7-yl)-3-(((2-amino-3-chloro-5-fluoroquinolin-7-yl)oxy)methyl)cyclopent-3-ene-1,2-diol;
(1S,2R,5R)-3-(2-(2-Amino-3-chloro-5-fluoroquinolin-7-yl)ethyl)-5-(6-amino-4-methyl-1H-pyrazolo[3,4-d]pyrimidin-1-yl)cyclopent-3-ene-1,2-diol;
(1S,2R,5R)-5-(4-Amino-2-methyl-7H-pyrrolo[2,3-d]pyrimidin-7-yl)-3-(((6-(difluoromethyl)-1,2,3,4-tetrahydroisoquinolin-8-yl)oxy)methyl)cyclopent-3-ene-1,2-diol;
(1S,2R,5R)-5-(4-Amino-2-methyl-7H-pyrrolo[2,3-d]pyrimidin-7-yl)-3-(2-(6-(difluoromethyl)-1,2,3,4-tetrahydroisoquinolin-8-yl)ethyl)cyclopent-3-ene-1,2-diol;
(1S,2R,5R)-5-(4-Amino-2-methyl-7H-pyrrolo[2,3-d]pyrimidin-7-yl)-3-(2-(2-amino-3-chloro-5-fluoroquinolin-7-yl)ethyl)cyclopent-3-ene-1,2-diol, and
(1S,2R,5R)-5-(2-Amino-4-methyl-7H-pyrrolo[2,3-d]pyrimidin-7-yl)-3-(2-(6-(difluoromethyl)-5-fluoro-1,2,3,4-tetrahydroiso quinolin-8-yl)ethyl) cyclopent-3-ene-1,2-diol.

### DETAILED DESCRIPTION OF THE INVENTION

Unless otherwise stated, the following terms used in the specification and claims have the meanings given below.

For purposes of interpreting the specification, the following definitions will apply and whenever appropriate, terms used in the singular will also include the plural and vice versa.

The terms “halogen” or “halo” means fluorine, chlorine, bromine, or iodine.

The term “alkyl” refers to an alkane derived hydrocarbon radical that includes solely carbon and hydrogen atoms in the backbone, contains no unsaturation, has from one to six carbon atoms, and is attached to the remainder of the molecule by a single bond, for example (C₁-C₆)alkyl or (C₁-C₄)alkyl, representative groups include e.g., methyl, ethyl, n-propyl, 1-methylethyl (isopropyl), n-butyl, n-pentyl and the like. Unless set forth or recited to the contrary, all alkyl groups described or claimed herein may be straight chain or branched.

The term “haloalkyl” refers to an alkyl group as defined above that is substituted by one or more halogen atoms as defined above. For example (C₁-C₆)haloalkyl or (C₁-C₄)haloalkyl. Suitably, the haloalkyl may be monohaloalkyl, dihaloalkyl or polyhaloalkyl including perhaloalkyl. A monohaloalkyl can have one iodine, bromine, chlorine or fluorine atom. Dihaloalkyl and polyhaloalkyl groups can be substituted with two or more of the same halogen atoms or a combination of different halogen atoms. Suitably, a polyhaloalkyl is substituted with up to 12 halogen atoms. Non-limiting Examples of a haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, dichloropropyl and the like. A perhaloalkyl refers to an alkyl having all hydrogen atoms replaced with halogen atoms. Unless set forth or recited to the contrary, all haloalkyl groups described or claimed herein may be straight chain or branched.

The term “alkoxy” denotes an alkyl group attached via an oxygen linkage to the rest of the molecule. Representative examples of such groups are —OCH₃ and —OC₂H₅. Unless set forth or recited to the contrary, all alkoxy groups described or claimed herein may be straight chain or branched.

The term “alkoxyalkyl” refers to an alkoxy group as defined above directly bonded to an alkyl group as defined above, e.g., —CH₂—O—CH₃, —CH₂—O—CH₂CH₃, —CH₂CH₂—O—CH₃ and the like.

The term “cycloalkyl” refers to a non-aromatic mono or multicyclic ring system having 3 to 12 carbon atoms, such as (C₃-C₁₀)cycloalkyl, (C₃-C₆)cycloalkyl, cyclopropyl, cyclobutyl, cyclopentyl, cyclohexyl and the like. Examples of multicyclic cycloalkyl groups include, but are not limited to, perhydronaphththyl, adamantyl and norbornyl groups, bridged cyclic groups or spirobicyclic groups, e.g., spiro(4,4)non-2-yl and the like.

The term “aryl” refers to an aromatic radical having 6- to 14-carbon atoms, including monocyclic, bicyclic and tricyclic aromatic systems, such as phenyl, naphthyl, tetrahydronaphthyl, indanyl, and biphenyl and the like.

The term “heterocyclic ring” or “heterocyclyl ring” or “heterocyclyl”, unless otherwise specified, refers to substituted or unsubstituted non-aromatic 3- to 15-membered ring which consists of carbon atoms and with one or more heteroatom(s) independently selected from N, O or S. The heterocyclic ring may be a mono-, bi- or tricyclic ring system, which may include fused, bridged or spiro ring systems and the nitrogen, carbon, oxygen or sulfur atoms in the heterocyclic ring may be optionally oxidized to various oxidation states. In addition, the nitrogen atom may be optionally quaternized, the heterocyclic ring or heterocyclyl may optionally contain one or more olefinic bond(S), and one or two carbon atoms(S) in the heterocyclic ring or heterocyclyl may be interrupted with —CF₂—, —C(O)—, —S(O)—, S(O)₂ etc. In addition, heterocyclic ring may also be fused with aromatic ring. Non-limiting Examples of heterocyclic rings include azetidinyl, benzopyranyl, chromanyl, decahydroisoquinolyl, indolinyl, isoindolinyl, isochromanyl, isothiazolidinyl, isoxazolidinyl, morpholinyl, oxazolinyl, oxazolidinyl, 2-oxopiperazinyl, 2-oxopiperidinyl, 2-oxopyrrolidinyl, 2-oxoazepinyl, octahydroindolyl, octahydroisoindolyl, perhydroazepinyl, piperazinyl, 4-piperidonyl, pyrrolidinyl, piperidinyl, phenothiazinyl, phenoxazinyl, quinuclidinyl, tetrahydroisquinolyl, tetrahydrofuryl, tetrahydropyranyl, thiazolinyl, thiazolidinyl, thiamorpholinyl, thiamorpholinylsulfoxide, thiamorpholinylsulfoneindoline, benzodioxole, tetrahydroquinoline, tetrahydrobenzopyran and the like. The heterocyclic ring may be attached by any atom of the heterocyclic ring that results in the creation of a stable structure.

The term “heteroaryl” unless otherwise specified, refers to a substituted or unsubstituted 5- to 14-membered aromatic heterocyclic ring with one or more heteroatom(S) independently selected from N, O or S. The heteroaryl may be a mono-, bi- or tricyclic ring system. The heteroaryl ring may be attached by any atom of the heteroaryl ring that results in the creation of a stable structure. Non-limiting Examples of a heteroaryl ring include oxazolyl, isoxazolyl, imidazolyl, furyl, indolyl, isoindolyl, pyrrolyl, triazolyl, triazinyl, tetrazolyl, thienyl, thiazolyl, isothiazolyl, pyridyl, pyrimidinyl, pyrazinyl, pyridazinyl, benzofuranyl, benzothiazolyl, benzoxazolyl, benzimidazolyl, benzothienyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indolizinyl, acridinyl, phenazinyl, phthalazinyl and the like.

The compounds of the present invention may have one or more chiral centers. The absolute stereochemistry at each chiral center may be ‘R’ or ‘S’. The compounds of the invention include all diastereomers and enantiomers and mixtures thereof. Unless specifically mentioned otherwise, reference to one stereoisomer applies to any of the possible stereoisomers. Whenever the stereoisomeric composition is unspecified, it is to be understood that all possible stereoisomers are included.

The term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are non-superimposable mirror images of one another. The term “chiral center” refers to a carbon atom to which four different groups are attached. As used herein, the term “diastereomers” refers to stereoisomers which are not enantiomers. The terms “racemate” or “racemic mixture” refer to a mixture of equal parts of enantiomers.

A “tautomer” refers to a compound that undergoes rapid proton shifts from one atom of the compound to another atom of the compound. Some of the compounds described herein may exist as tautomers with different points of attachment of hydrogen. The individual tautomers as well as mixture thereof are encompassed with compounds of formula (I).

The term “treating” or “treatment” of a state, disorder or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; c) lessening the disease, disorder or condition or at least one of its clinical or subclinical symptoms or (d) relieving the disease, i.e., causing regression of the state, disorder or condition or at least one of its clinical or subclinical symptoms.

The term “inhibitor” refers to a molecule that binds to an enzyme to inhibit the activity of the said enzyme either partially or completely.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a disease, disorder or condition, is sufficient to cause the effect in the subject, which is the purpose of the administration. The “therapeutically effective amount” will vary depending on the compound, the disease and its severity and the age, weight, physical condition, and responsiveness of the subject to be treated.

### Pharmaceutically Acceptable Salts

The compounds of the invention may form salts with acid or base. The compounds of invention may be sufficiently basic or acidic to form stable nontoxic acid or base salts, administration of the compound as a pharmaceutically acceptable salt may be appropriate. Non-limiting Examples of pharmaceutically acceptable salts are inorganic, organic acid addition salts formed by addition of acids including hydrochloride salts. Non-limiting Examples of pharmaceutically acceptable salts are inorganic, organic base addition salts formed by addition of bases. The compounds of the invention may also form salts with amino acids. Pharmaceutically acceptable salts may be obtained using standard procedures well known in the art, for example by reacting sufficiently basic compound such as an amine with a suitable acid.

Screening of the compounds of invention for PRMT5 inhibitory activity can be achieved by using various in vitro and in vivo protocols mentioned herein below or methods known in the art.

### Pharmaceutical Compositions

The invention relates to pharmaceutical compositions containing the compounds of the formula (I), or pharmaceutically acceptable salts thereof disclosed herein. In particular, pharmaceutical compositions containing a therapeutically effective amount of at least one compound of formula (I) described herein and at least one pharmaceutically acceptable excipient (such as a carrier or diluent). Preferably, the contemplated pharmaceutical compositions include the compound(s) described herein in an amount sufficient to inhibit PRMT5 to treat the diseases described herein when administered to a subject.

The subjects contemplated include, for example, a living cell and a mammal, including human. The compound of the invention may be associated with a pharmaceutically acceptable excipient (such as a carrier or a diluent) or be diluted by a carrier, or enclosed within a carrier which can be in the form of a capsule, sachet, paper or other container. The pharmaceutically acceptable excipient includes pharmaceutical agent that does not itself induce the production of antibodies harmful to the individual receiving the composition, and which may be administered without undue toxicity.

Examples of suitable carriers or excipients include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, salicylic acid, fatty acids, fatty acid amines, fatty acid monoglycerides and diglycerides, pentaerytritol fatty acid esters, polyoxyethylene, hydroxymethylcellulose and polyvinylpyrrolidone.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing procedures known in the art.

The pharmaceutical compositions described herein may be prepared by conventional techniques known in the art. For example, the active compound can be mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound can be adsorbed on a granular solid container, for Example, in a sachet.

The pharmaceutical compositions may be in conventional forms, for example, capsules, tablets, caplets, orally disintegrating tablets, aerosols, solutions, suspensions, or products for topical application.

The route of administration may be any route which effectively transports the active compound of the invention to the appropriate or desired site of action. Suitable routes of administration include, but are not limited to, oral, oral inhalation, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment).

Solid oral formulations include, but are not limited to, tablets, caplets, capsules (soft or hard gelatin), orally disintegrating tablets, dragees (containing the active ingredient in powder or pellet form), troches and lozenges. Tablets, dragees, or capsules having talc and/or a carbohydrate carrier or binder or the like are particularly suitable for oral application. Liquid formulations include, but are not limited to, syrups, emulsions, suspensions, solutions, soft gelatin and sterile injectable liquids, such as aqueous or non-aqueous liquid suspensions or solutions. For parenteral application, particularly suitable are injectable solutions or suspensions, preferably aqueous solutions with the active compound dissolved in polyhydroxylated castor oil.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as pocketed tablets, capsules, and powders in vials or ampoules. Also, the unit dosage form can be a capsule, tablet, caplet, cachet, or lozenge itself, or it can be the appropriate number of any of these in packaged form.

For administration to subject patients, the total daily dose of the compounds of the invention depends, of course, on the mode of administration. For example, oral administration may require a higher total daily dose, than an intravenous (direct into blood). The quantity of active component in a unit dose preparation may be varied or adjusted from 0.1 mg to 1000 mg by oral administration and 1 μg to 5000 μg by inhalation according to the potency of the active component or mode of administration.

Those skilled in the relevant art can determine suitable doses of the compounds for use in treating the diseases and disorders described herein. Therapeutic doses are generally identified through a dose ranging study in subject based on preliminary evidence derived from the animal studies. Doses must be sufficient to result in a desired therapeutic benefit without causing unwanted side effects for the patient. For example, the daily dosage of the PRMT5 inhibitor can range from about 0.1 to about 30.0 mg/kg by oral administration. Mode of administration, dosage forms, suitable pharmaceutical excipients, diluents or carriers can also be well used and adjusted by those skilled in the art. All changes and modifications envisioned are within the scope of the invention.

The invention provides compound of formula (I) and pharmaceutical compositions thereof as protein arginine methyl transferase-5 (PRMT5) inhibitors for treating the diseases, disorders or conditions associated with overexpression of PRMT5. The invention further provides a method of treating diseases, disorders or conditions associated with overexpression of PRMT5 in a subject in need thereof by administering to the subject a therapeutically effective amount of a compound or a pharmaceutical composition of the invention.

In another aspect, the invention relates to a method of treating diseases, disorders or conditions associated with the overexpression of PRMT5. In this method, a subject in need of such treatment is administered a therapeutically effective amount of a compound of formula (I) or a pharmaceutically acceptable salt thereof as described herein.

In one embodiment of the present invention, the diseases, disorders, or conditions associated with the overexpression of PRMT5 are cancer.

In another embodiment, the invention provides a method of treating cancers, particularly, glioblastoma multiforme, prostate cancer, pancreatic cancer, mantle cell lymphoma, non-Hodgkin's lymphomas and diffuse large B-cell lymphoma, acute myeloid leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, and cervical cancer.

It is to be understood that the invention encompasses the compounds of formula (I) or pharmaceutically acceptable salts thereof for use in the treatment of a disease or disorder mentioned herein.

It is to be understood that the invention encompasses the compounds of formula (I) or pharmaceutically acceptable salts thereof in the manufacture of a medicament for treating a disease or disorder mentioned herein.

A compound, its tautomeric form, its stereoisomer, its pharmaceutically acceptable salt, its polymorph, its solvate, its combination with suitable medicament, its pharmaceutical composition thereof as described hereinabove wherein the compound of general formula (I), is selected from the group consisting of:
(1S,2R,5R)-3-(2-(2-Amino-3-chloro-5-fluoroquinolin-7-yl)ethyl)-5-(2-amino-4-methyl-7H-pyrrolo[2,3-d]pyrimidin-7-yl)cyclopent-3-ene-1,2-diol;
(1S,2R,5R)-3-(((2-Amino-3-chloro-5-fluoroquinolin-7-yl)oxy)methyl)-5-(2-amino-4-methyl-7H-pyrrolo[2,3-d]pyrimidin-7-yl)cyclopent-3-ene-1,2-diol;
(1S,2R,5R)-5-(4-Amino-2-methyl-7H-pyrrolo[2,3-d]pyrimidin-7-yl)-3-(((2-amino-3-chloro-5-fluoroquinolin-7-yl)oxy)methyl)cyclopent-3-ene-1,2-diol;
(1S,2R,5R)-3-(2-(2-Amino-3-chloro-5-fluoroquinolin-7-yl)ethyl)-5-(6-amino-4-methyl-1H-pyrazolo[3,4-d]pyrimidin-1-yl)cyclopent-3-ene-1,2-diol;
(1S,2R,5R)-5-(4-Amino-2-methyl-7H-pyrrolo[2,3-d]pyrimidin-7-yl)-3-(((6-(difluoromethyl)-1,2,3,4-tetrahydroisoquinolin-8-yl)oxy)methyl)cyclopent-3-ene-1,2-diol;
(1S,2R,5R)-5-(4-Amino-2-methyl-7H-pyrrolo[2,3-d]pyrimidin-7-yl)-3-(2-(6-(difluoromethyl)-1,2,3,4-tetrahydroisoquinolin-8-yl)ethyl)cyclopent-3-ene-1,2-diol;
(1S,2R,5R)-5-(4-Amino-2-methyl-7H-pyrrolo[2,3-d]pyrimidin-7-yl)-3-(2-(2-amino-3-chloro-5-fluoroquinolin-7-yl)ethyl)cyclopent-3-ene-1,2-diol and
(1S,2R,5R)-5-(2-Amino-4-methyl-7H-pyrrolo[2,3-d]pyrimidin-7-yl)-3-(2-(6-(difluoromethyl)-5-fluoro-1,2,3,4-tetrahydroiso quinolin-8-yl)ethyl)cyclopent-3-ene-1,2-diol
   or pharmaceutically acceptable salt thereof.

The compound of formula described herein may be prepared by techniques known in the art. In addition, the compound of formula described herein may be prepared by following the reaction sequence as depicted in Schemes provided below. Further, in the following schemes, where specific bases, acids, reagents, solvents, coupling agents, etc., are mentioned, it is understood that other bases, acids, reagents, solvents, coupling agents etc., known in the art may also be used and are therefore included within the scope of the present invention. Variations in reaction conditions, for example, temperature and/or duration of the reaction, which may be used as known in the art, are also within the scope of the present invention. All the isomers of the compound of formula in described in these schemes, unless otherwise specified, are also encompassed within the scope of this invention.

Scheme-1 illustrates the synthesis of compound of formula 11. Compound of formula 1 (where PG=Protecting group such as but not limited to TBDPS, TBDMS etc.), is prepared by following the procedure described in Purinergic Signaling (2015) 11:371-387. Mitsunobu reaction of compound of formula 1 with compound of formula 2 (X═—Cl, —Br) using various azo dicarboxylate reagents such as but not limited to DEAD or DIAD in presence of phosphine such as but not limited to PPh₃ gives the compound of formula 3. Typically, these reactions are run in ethereal solvents such as THF, MeTHF, dioxane, or similar solvents at temperatures ranging from 0° C. to 25° C. Compound of formula 4 is formed upon treatment of compound of formula 3 with fluoride ions such as but not limited to TBAF. Typically these reactions are done in ethereal solvents such as THF, MeTHF, dioxane, or similar solvents at temperatures ranging from 0° C. to 40° C. Oxidation of compound of formula 4 with various oxidizing agents such as but not limited to Dess-Martin periodinane can furnish the compound of formula 5. Typically these reactions can be run in halogenated solvents such as CH₂Cl₂, CHCl₃, dichloroethane or similar solvents at temperatures ranging from 0° C. to 40° C. Reagents such as but not limited to methyltriphenylphosphonium bromide in presence of a base such as but not limited to KO^{t}Bu, NaO^{t}Bu, LiHMDS, NaHMDS, or KHMDS when treated with compound of formula 5 affords compound of formula 6. Typically these reactions are done in ethereal solvents such as THF, MeTHF, dioxane, or similar solvents at temperatures ranging from 0° C. to 25° C. Compound of formula 8 can be synthesized by hydroboration of compound of formula 6 with suitable boranes such as but not limited to 9-BBN followed by addition of inorganic base such as but not limited to tripotassium phosphate or Cs₂CO₃, in presence of Pd catalyst such as but not limited to Pd(dppf)Cl₂ or Pd-118 and compound of formula 7 (Y═—Br, —I), which was synthesized by following the procedure reported in Journal of the American Chemical Society, 1949, vol. 71, p. 6,7. Typically these reactions are done in etheral solvents such as THF, MeTHF, dioxane, or similar solvents at temperatures ranging from 25° C. to 70° C.

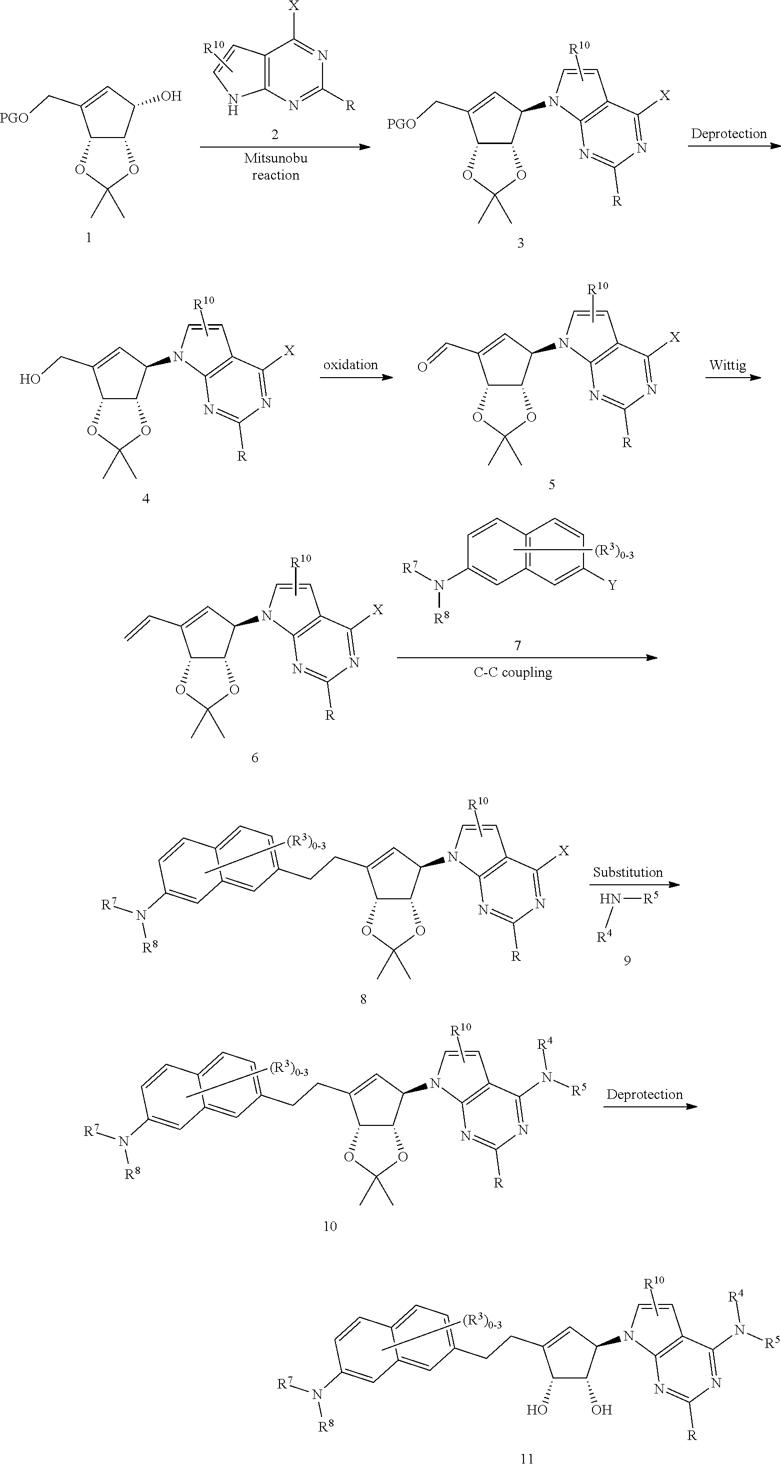

Compound of formula 9 (where R⁴ and R⁵ is defined herein above) upon treatment with compound of formula 8 affords compound of formula 10. Acetonide deprotection of compound of formula 10 with acids such as but not limited to HCl or TFA affords compound of formula 11. Typically, these reactions are run at temperatures ranging from 25° C. to 50° C. Compounds at every step may be purified by standard techniques such as column chromatography, crystallization, reverse phase HPLC, or SFC.

Scheme-2 illustrates the synthesis of compound of formula 16. Iodination of compound of formula 4 with various reagents such as but not limited to Iodine/PPh₃/Imidazole can furnish the compound of formula 12. Typically these reactions can be run in halogenated solvents such as but not limited to CH₂Cl₂ at temperatures ranging from 0° C. to 25° C. for about 30 mins to 3 h. Base such as but not limited to Cs₂CO₃ when treated with compound of formula 12 in presence of compound of formula 13 (synthesis is described herein below in scheme-7; PG₁=-Boc) affords compound of formula 14. Typically, these reactions are done in solvents such as DMF DMAc, or similar solvents at temperatures ranging from 0° C. to 25° C. for about 30 mins to 3 h.

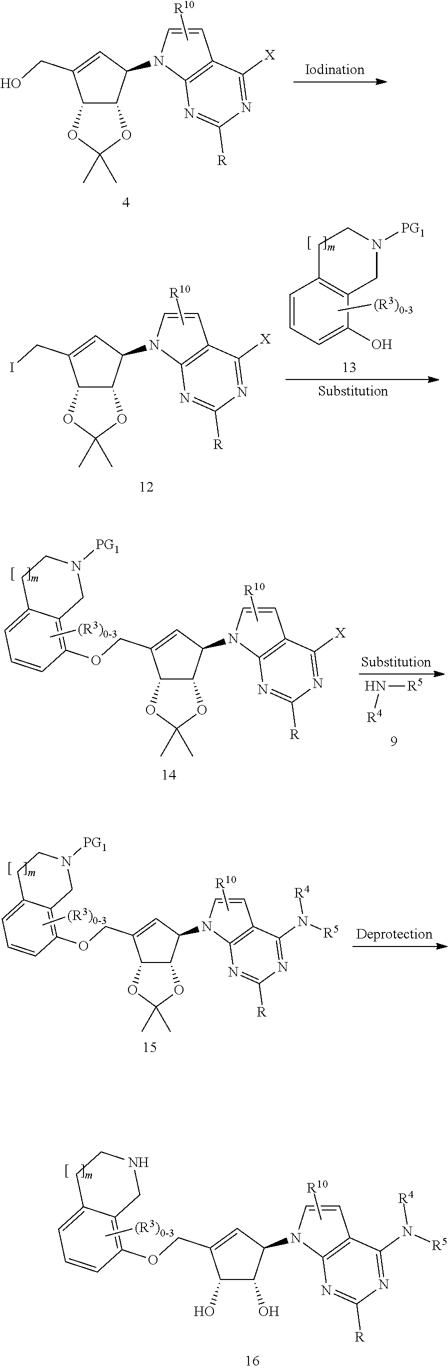

Compound of formula 9 (where R⁴ and R⁵ is defined herein above) upon treatment with compound of formula 14 affords compound of formula 15. Typically, these reactions are done in ethereal solvents such as dioxane, or similar solvents at temperatures ranging from 100° C. to 140° C. for about 8-16 h. Deprotection of compound of formula 15 with acids such as but not limited to HCl or TFA affords compound of formula 16. Typically these reactions are run at temperatures ranging from 25° C. to 50° C. for about 1-5 h. Compounds at every step may be purified by standard techniques such as column chromatography, crystallization, reverse phase HPLC, or SFC.

Scheme-3 illustrates the synthesis of compound of formula 20. Compound of formula 18 can be synthesized by hydroboration of compound of formula 6 with suitable boranes such as but not limited to 9-BBN followed by addition of inorganic base such as but not limited to tripotassium phosphate in presence of Pd catalyst such as but not limited to Pd(dppf)Cl₂ or Pd-118 and compound of formula 17 (synthesis is described herein below in scheme-7; Y═—Br, —I; PG₁=-Boc). Typically, these reactions are done in ethereal solvents such as THF, MeTHF, dioxane, or similar solvents at temperatures ranging from 25° C. to 70° C. for about 1-8 h.

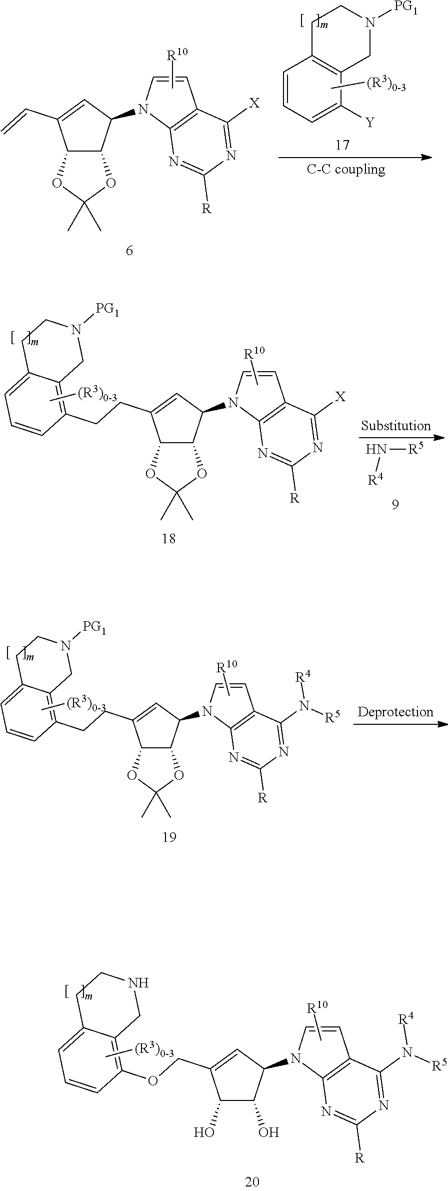

Compound of formula 9 (where R⁴ and R⁵ is defined herein above) upon treatment with compound of formula 18 affords compound of formula 19. Typically, these reactions are done in ethereal solvents such as dioxane or similar solvents at temperatures ranging from 100° C. to 140° C. for about 8-16 h. Deprotection of compound of formula 19 with acids such as but not limited to HCl or TFA affords compound of formula 20. Typically, these reactions are run at temperatures ranging from 25° C. to 50° C. for about 1-5 h. Compounds at every step may be purified by standard techniques such as column chromatography, crystallization, reverse phase HPLC, or SFC.

Scheme-4 illustrates the synthesis of compound of formula 28. Oxidation of compound of formula 21 (which was synthesized as described in Bio-organic and medicinal Chemistry, 1996, 4(7), 1077-1088) with various oxidizing agents such as but not limited to Dess-Martin periodinane can furnish the compound of formula 22. Typically, these reactions can be run in halogenated solvents such as CH₂Cl₂, CHCl₃, dichloroethane or similar solvents at temperatures ranging from 0° C. to 25° C. for about 1-3 h. Reagents such as but not limited to methyltriphenylphosphonium bromide in presence of a base such as but not limited to n-BuLi when treated with compound of formula 22 provides compound of formula 23. Typically these reactions are done in ethereal solvents such as THF, MeTHF, dioxane, or similar solvents at temperatures ranging from 0° C. to 10° C. for about 10 min to 2 h. Compound of formula 24 can be synthesized by hydroboration of compound of formula 23 with suitable boranes such as but not limited to 9-BBN followed by addition of inorganic base such as but not limited to tripotassium phosphate, in presence of Pd catalyst such as but not limited to Pd(dppf)Cl₂ or Pd-118 and compound of formula 7 (Y═—Br, —I), which was synthesized by following the procedure reported in Journal of the American Chemical Society, 1949, vol. 71, p. 6,7. Typically these reactions are done in ethereal solvents such as THF, MeTHF, dioxane, or similar solvents at temperatures ranging from 0° C. to 25° C. for about 1 h to 4 h.

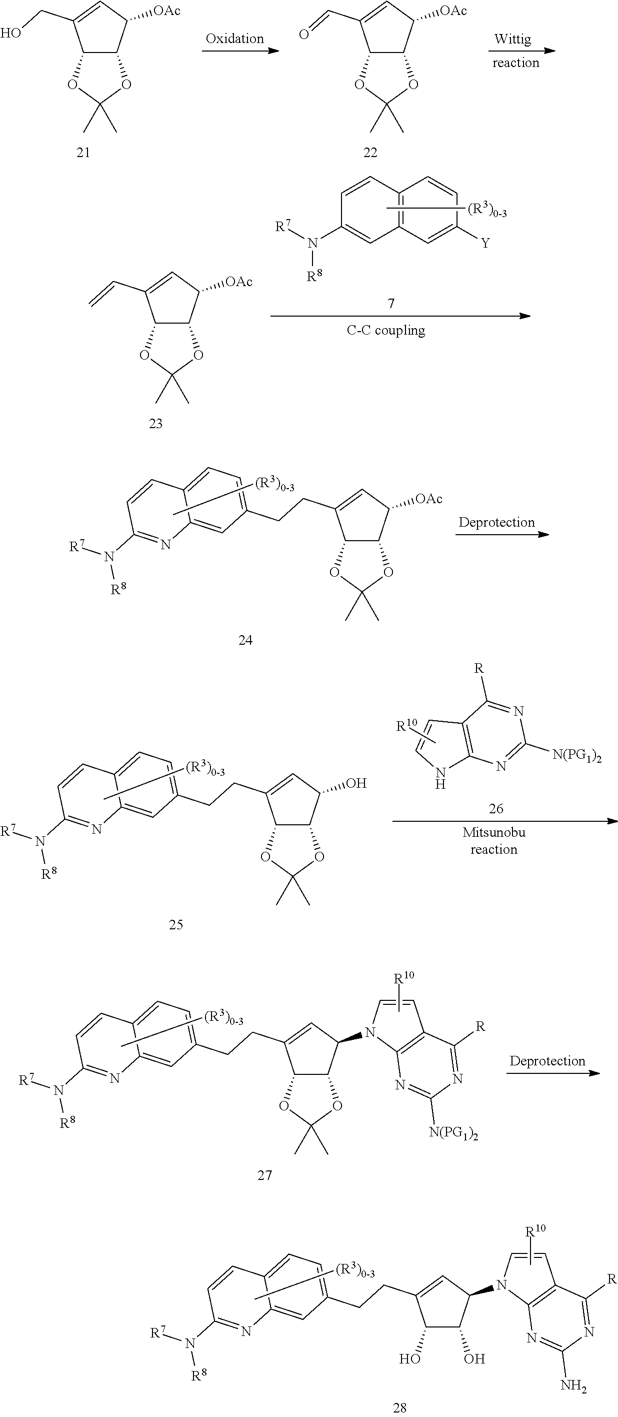

Deprotection of compound of formula 24 with bases such as but not limited to K₂CO₃ or Na₂CO₃ affords compound of formula 25. Typically these reactions are run in protic solvents such as but not limited to MeOH or EtOH at temperatures ranging from 0° C. to 25° C. for about 1 h to 3 h. Mitsunobu reaction of compound of formula 25 with compound of formula 26 (PG₁=-Boc) using various azodicarboxylate reagents such as but not limited to DEAD or DIAD in presence of phosphine such as but not limited to PPh₃ gives the compound of formula 27. Typically, these reactions are run in ethereal solvents such as THF, MeTHF, dioxane, or similar solvents at temperatures ranging from 0° C. to 25° C. for about 8 h to 48 h. Deprotection of compound of formula 27 with acids such as but not limited to HCl or TFA affords compound of formula 28. Typically, these reactions are run at temperatures ranging from 25° C. to 60° C. for about 1 h to 6 h. Compounds at every step may be purified by standard techniques such as column chromatography, crystallization, reverse phase HPLC, or SFC.

Scheme-5 illustrates the synthesis of compound of formula 33.

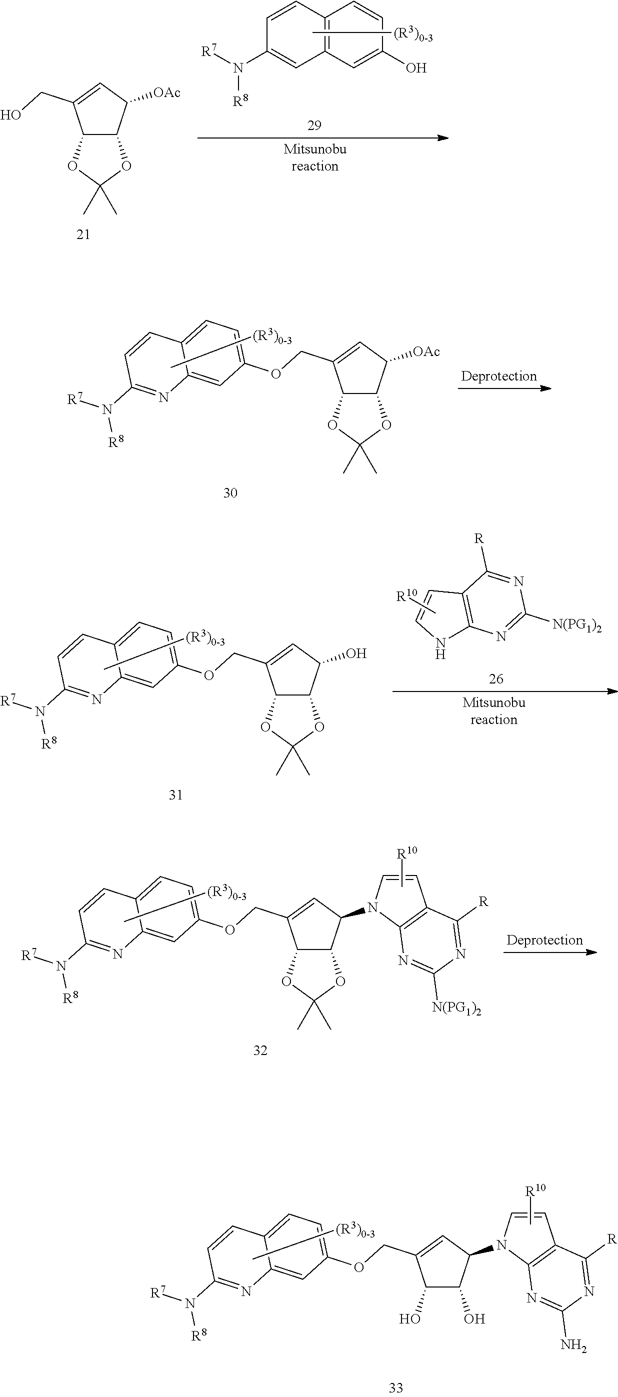

Mitsunobu reaction of compound of formula 21 with compound of formula 29 using various azodicarboxylate reagents such as but not limited to DEAD or DIAD in presence of phosphine such as but not limited to PPh₃ gives the compound of formula 30. Typically, these reactions are run in ethereal solvents such as THF, MeTHF, dioxane, or similar solvents at temperatures ranging from 0° C. to 25° C. for about 8 h to 16 h. Deprotection of compound of formula 30 with bases such as but not limited to K₂CO₃ or Na₂CO₃ affords compound of formula 31. Typically these reactions are run in protic solvents such as but not limited to MeOH or EtOH at temperatures ranging from 0° C. to 25° C. for about 1 h to 3 h. Mitsunobu reaction of compound of formula 31 with compound of formula 26 (PG₁=-Boc) using various azo dicarboxylate reagents such as but not limited to DEAD or DIAD in presence of phosphine such as but not limited to PPh₃ gives the compound of formula 32. Typically, these reactions are run in ethereal solvents such as THF, MeTHF, dioxane, or similar solvents at temperatures ranging from 0° C. to 25° C. for about 8 h to 16 h. Deprotection of compound of formula 32 with acids such as but not limited to HCl or TFA affords compound of formula 33. Typically, these reactions are run at temperatures ranging from 25° C. to 60° C. for about 1 h to 6 h. Compounds at every step may be purified by standard techniques such as column chromatography, crystallization, reverse phase HPLC, or SFC.

Scheme-6 illustrates the synthesis of compound of formula 36.

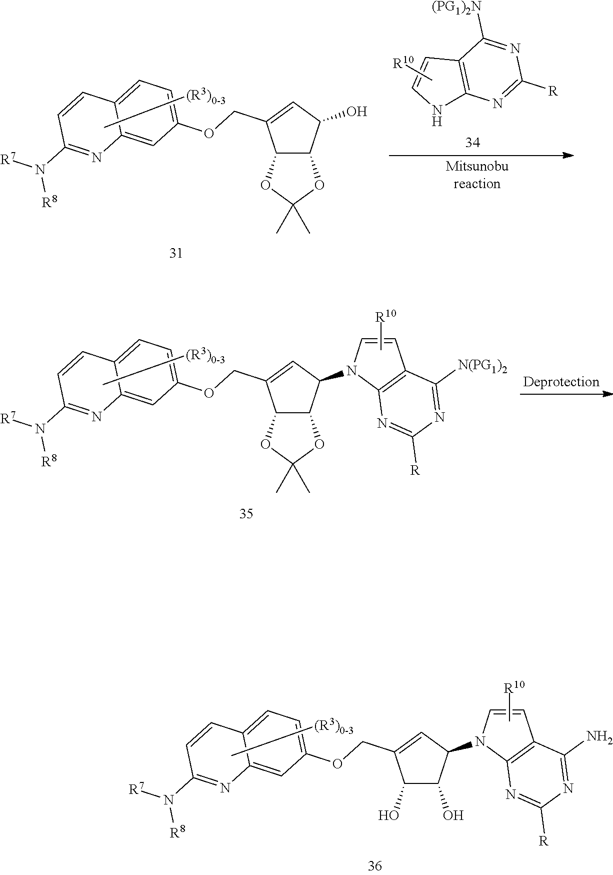

Mitsunobu reaction of compound of formula 31 with compound of formula 34 (PG₁=-Boc) using various azo dicarboxylate reagents such as but not limited to DEAD or DIAD in presence of phosphine such as but not limited to PPh₃ gives the compound of formula 35. Typically, these reactions are run in ethereal solvents such as THF, MeTHF, dioxane, or similar solvents at temperatures ranging from 0° C. to 25° C. for about 8 h to 16 h. Deprotection of compound of formula 35 with acids such as but not limited to HCl or TFA affords compound of formula 36. Typically these reactions are run at temperatures ranging from 25° C. to 60° C. for about 1 h to 6 h. Compounds at every step may be purified by standard techniques such as column chromatography, crystallization, reverse phase HPLC, or SFC.

Scheme-7 illustrates the synthesis of compound of formula 48. Treating compound of formula 37 with compound of formula 37a affords compound of formula 38. Typically, these reactions are run in solvent such as but not limited to toluene at temperatures ranging from 100° C. to 120° C. for about 1 h to 5 h.

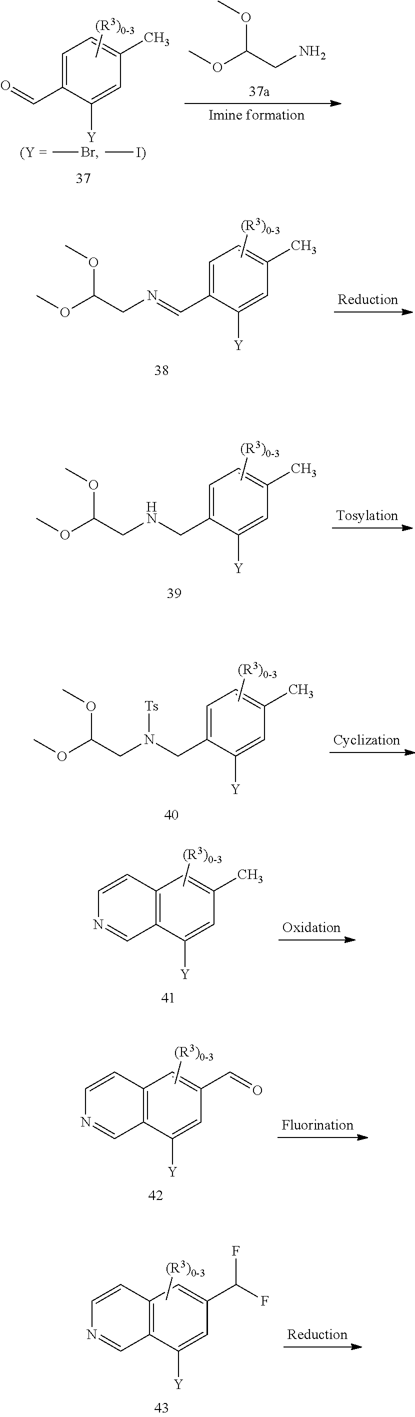

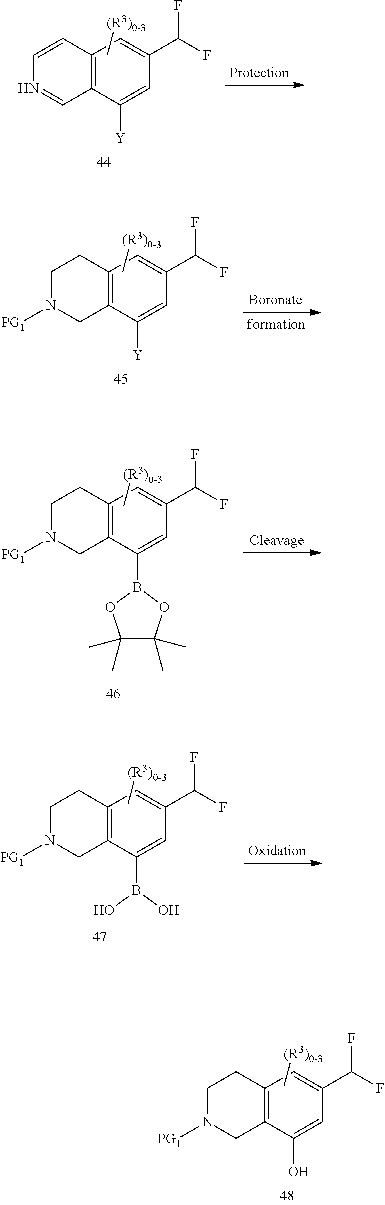

Reduction of compound of formula 38 with reducing agent such as but not limited to NaBH₄ gives compound of formula 39. Typically, these reactions are run in protic solvents such as but not limited to MeOH or EtOH at temperatures ranging from 0° C. to 25° C. for about 1 h to 3 h. Tosylation of compound of formula 39 with reagents such as but not limited to TsCl/Pyridine provides compound of formula 40. Typically, these reactions are run in halogenated solvents such as but not limited to CH₂Cl₂ at temperatures ranging from 0° C. to 25° C. for about 5 h to 16 h. Cyclization of compound of formula 40 with reagents such as but not limited to AlCl₃ affords compound of formula 41. Typically, these reactions are run in halogenated solvents such as but not limited to CH₂Cl₂ at temperatures ranging from 0° C. to 25° C. for about 5 h to 16 h. Oxidation of compound of formula 41 with oxidizing agents such as but not limited to selenium dioxide gives compound of formula 42. Typically, these reactions are run in hydrocarbon solvents such as but not limited to 1,2-dichlorobenzene at temperatures ranging from 150° C. to 180° C. for about 2 h to 8 h. Fluorination of compound of formula 42 with fluorinating agents such as but not limited to DAST gives compound of formula 43. Typically, these reactions are run in halogenated solvents such as but not limited to CH₂Cl₂ at temperatures ranging from 0° C. to 25° C. for about 5 h to 16 h. Reduction of compound of formula 43 with reducing agent such as but not limited to NaBH₄ gives compound of formula 44. Typically, these reactions are run in solvents such as but not limited to acetic acid at temperatures ranging from 0° C. to 25° C. for about 1 h to 3 h. Protection of compound of formula 44 with reagents such as but not limited to Boc-anhydride/NEts gives compound of formula 45. Typically, these reactions are run in halogenated solvents such as but not limited to CH₂Cl₂ at temperatures ranging from 0° C. to 25° C. for about 1 h to 3 h. Compound of formula 46 can be synthesized by treating compound of formula 45 with suitable boron reagents such as but not limited to bispinacoloto diboron followed by addition of inorganic base such as but not limited to potassium acetate, in presence of Pd catalyst such as but not limited to Pd(dppf)Cl₂. Typically these reactions are done in ethereal solvents such as dioxane, or similar solvents at temperatures ranging from 100° C. to 110° C. for about 1 h to 4 h. Compound of formula 47 can be synthesized by treating compound of formula 46 with suitable reagents such as but not limited to sodium per iodate followed by addition of inorganic acid such as but not limited to HCl. Typically these reactions are done in solvents such as acetone, or similar solvents at temperatures ranging from 0° C. to 25° C. for about 1 h to 4 h. Oxidation of compound of formula 47 with oxidizing agents such as but not limited to hydrogen peroxide in presence of acid such as but not limited to citric acid gives compound of formula 48. Typically, these reactions are run at temperatures ranging from 0° C. to 25° C. for about 1 h to 3 h.

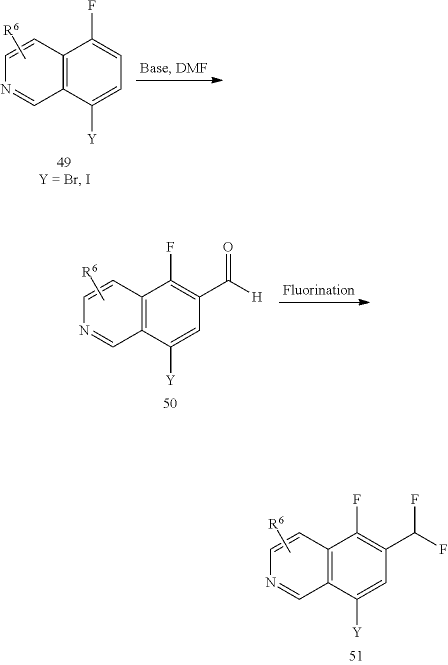

Scheme-8 illustrates the synthesis of compound of formula 51. Formylation reaction of compound of formula 49 with formylating agent such as but not limited to DMF in presence of hindered base such as but not limited to LDA gives the compound of formula 50. Typically these reactions are run in etheral solvents such as THF, MeTHF, dioxane, or similar solvents at temperatures ranging from −78° C. to 0° C. Compound of formula 50 upon treatment with nucleophilic fluorinating agents such as but not limited to DAST can give compound of formula 51. Typically, these reactions can be run in halogenated solvents such as CH₂Cl₂, CHCl₃ or similar solvents at temperatures ranging from 0° C. to 25° C.

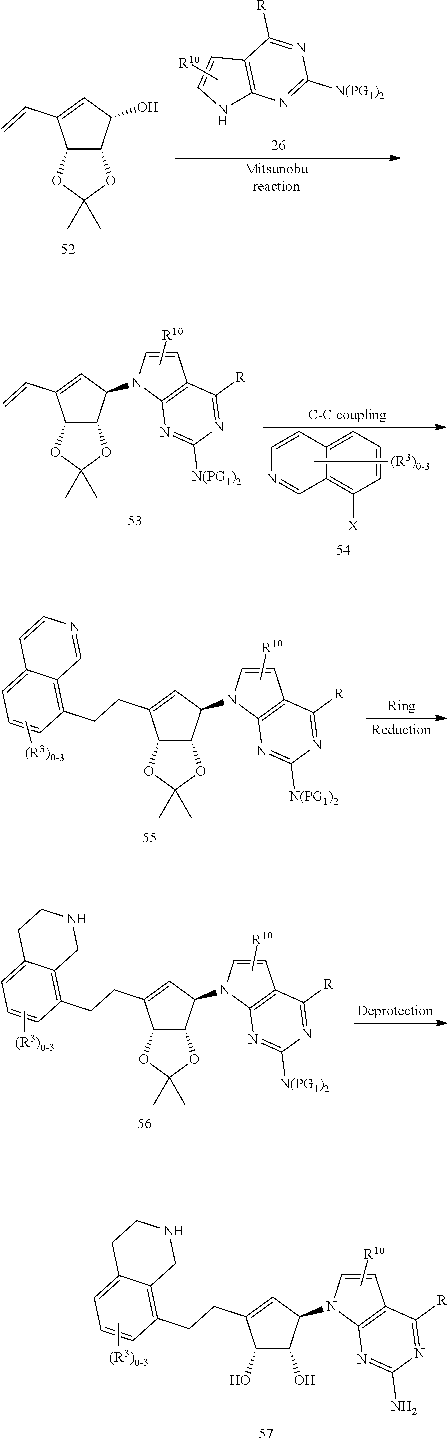

Scheme-9 illustrates the synthesis of compound of formula 57. Mitsunobu reaction of compound of formula 52 with compound of formula 26 (PG₁=-Boc) using various azodicarboxylate reagents such as but not limited to DEAD or DIAD in presence of phosphine such as but not limited to PPh₃ gives the compound of formula 53. Typically these reactions are run in etheral solvents such as for example THF, MeTHF, dioxane, or similar solvents at temperatures ranging from 0° C. to 25° C. Compound of formula 55 can be synthesized by hydroboration of compound of formula 53 with suitable boranes such as but not limited to 9-BBN followed by addition of inorganic base such as but not limited to tripotassium phosphate or Cs₂CO₃, in presence of Pd catalyst such as but not limited to Pd(dppf)Cl₂ or Pd-118 and compound of formula 54 (Y═Br, —I). Typically, these reactions are done in etheral solvents such as THF, MeTHF, dioxane, or similar solvents and run at temperatures ranging from 25° C. to 70° C. Compound of formula 55 upon treatment with reducing agents such as but not limited to NaBH₄ affords compound of formula 56. Typically, these reactions are done in acidic solvents such as for example acetic acid at temperatures ranging from 0° C. to 25° C. Additionally, deprotection of compound of formula 56 with acids such as but not limited to HCl or TFA affords compound of formula 57. Typically, these reactions are run at temperatures ranging from 25° C. to 50° C. Compounds at every step may be purified by standard techniques such as column chromatography, crystallization, reverse phase HPLC or SFC.

### ABBREVIATIONS

The following abbreviations may be used herein:
AcOH=Acetic acid
Aq.=aqueous
9-BBN=9-Borabicyclononane
Boc=tert-Butoxycarbonyl
t-Bu or tBu=tert-Butyl
Cs₂CO₃=Cesium Carbonate
CHCl₃=Chloroform
DAST=Diethylaminosulphur trifluoride
CH₂Cl₂ or DCM=Dichloromethane
DMP=Dess Martin Periodinane
DEAD=Diethyl azodicarboxylate
DIAD=Diisopropyl azodicarboxylate
DMAP=4-Dimethylaminopyridine
DMF=NN-Dimethylformamide
DMSO=Dimethylsulphoxide
DMSO-d₆=Deuterated dimethylsulphoxide
Et=ethyl
EtOH=Ethanol
EtOAc=Ethyl acetate
g=gram
K₂CO₃=Potassium carbonate
KOH=Potassium hydroxide
KO^{t}Bu=Potassium tert-butoxide
KHMDS=Potassium bis(trimethylsilyl)amide
LCMS=Liquid chromatography mass spectrometry
mg=milligram
Me=Methyl
MeOH=Methanol
MeOD=Deuterated methanol
MsCl=Methanesulphonyl chloride
MgSO₄=Magnesium sulphate
NaH=Sodium hydride
NaO^{t}Bu=Sodium tert-butoxide
NaHCO₃=Sodium bicarbonate
Na₂SO₄=Sodium sulphate
Na₂S₂O₃=Sodium thiosulphate
Na₂SO₃=Sodium sulphite
NaHMDS=Sodium bis(trimethylsilyl)amide
NMR=Nuclear magnetic resonance
Ph=phenyl
Pd-118=[1,1′-Bis(di-tert-butylphosphino)ferrocene]dichloropalladium(II)
PdCl₂(dppf)=[1,1′-Bis(diphenylphosphino)ferrocene]dichloropalladium(II)
PCC=Pyridinium chlorochromate
PMB=p-Methoxybenzyl
PTSA=p-Toluenesulphonic acid
Rt=Retention time
rt=room temperature
Sat.=saturated
SFC=Supercritical fluid chromatography
TLC=Thin layer chromatography
TBAF=Tetrabutylammonium fluoride
TsCl=p-Toluenesulphonyl chloride
TBDMS=tert-Butyldimethylsilyl
TBDPS=tert-Butyldiphenylsilyl
Et₃N or NEts or TEA=Trimethylamines
TFA=Trifluoroacetic acid
THF=Tetrahydrofuran
Ts=p-Toluenesulphonyl

### Intermediates

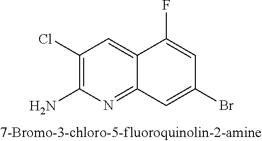 [SMILES: Nc1nc2cc(Br)cc(F)c2cc1Cl]

The title compound was prepared by following an analogous reaction protocol as described in WO2019/116302 A1.

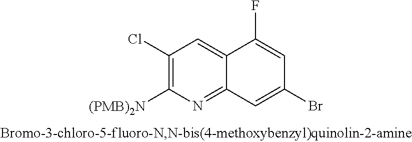

To a stirred suspension of 7-bromo-3-chloro-5-fluoroquinolin-2-amine (7.0 g, 25.4 mmol) in DMF (90 mL) was added sodium hydride (2.54 g, 63.5 mmol) at 0° C. The resulting mixture was stirred at 0° C. for 15 min. 1-(Chloromethyl)-4-methoxybenzene (8.27 mL, 61.0 mmol) was added dropwise under N₂ atmosphere. The reaction mixture was then stirred for 5 h at 25° C. The resulting mixture was poured onto ice water (100 mL). The solid precipitated out was filtered, washed with water (200 ml) and dried under vacuum. It was further co-evaporated with toluene and dried in vacuo to afford 7-bromo-3-chloro-5-fluoro-N,N-bis(4-methoxybenzyl)quinolin-2-amine (13 g, 99%) as an off white solid. ¹H NMR (400 MHz, DMSO-d6) δ 8.39 (d, J=0.9 Hz, 1H), 7.76 (t, J=1.3 Hz, 1H), 7.52 (dd, J=9.4, 1.7 Hz, 1H), 7.29-7.23 (m, 4H), 6.91-6.84 (m, 4H), 4.60 (s, 4H), 3.71 (s, 6H); LCMS m/z=514.68, 516.68 (M+, M+2; 100%).

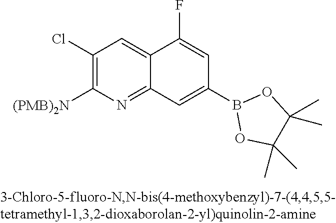

A mixture of 7-bromo-3-chloro-5-fluoro-N,N-bis(4-methoxybenzyl)quinolin-2-amine (2.200 g, 4.27 mmol), PdCl₂(dppf) (0.312 g, 0.427 mmol), bis(pinacolato)diboron (1.3 g, 5.12 mmol), potassium acetate (0.712 g, 7.25 mmol) and DMSO (5 ml) was heated at 80° C. for 30 min. in preheated oil bath. The resulting mixture was slowly poured onto ice cold water (50 ml) and extracted with ethyl acetate (50 ml). Layers were separated, and the organic layer was washed with brine (50 ml) and dried over anhydrous Na₂SO₄. The organic layer was filtered and concentrated in vacuo to give 2.5 g of crude compound. This residue was purified by combiflash (R_{f}200, Teledyne/Isco) instrument onto a redisep® R_{f}column with gradient elution (0 to 10%) of ethyl acetate in petroleum ether to afford 3-chloro-5-fluoro-N,N-bis(4-methoxybenzyl)-7-(4,4,5,5-tetramethyl-1,3,2-dioxaborolan-2-yl)quinolin-2-amine (2 g, 83%) as an off white solid. ¹H NMR (400 MHz, DMSO-d6) δ 8.44 (d, J=0.9 Hz, 1H), 7.81 (t, J=0.9 Hz, 1H), 7.31-7.28 (m, 1H), 7.27-7.23 (m, 4H), 6.89-6.84 (m, 4H), 4.59 (d, J=12.3 Hz, 4H), 3.70 (s, 6H), 1.17 (s, 12H).

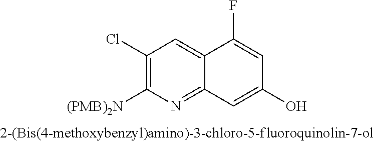

To a stirred solution of 3-chloro-5-fluoro-N,N-bis(4-methoxybenzyl)-7-(4,4,5,5-tetramethyl-1,3,2-dioxaborolan-2-yl)quinolin-2-amine (1.6 g, 2.84 mmol) in THF (35 ml), was added glacial acetic acid (0.325 ml, 5.69 mmol) dropwise at 0° C. The mixture was stirred at 0° C. for 1 h. An aq. solution of hydrogen peroxide (1.742 ml, 17.06 mmol) was added slowly and stirred the reaction mixture for 16 h at 25° C. The reaction mixture was diluted with ethyl acetate (50 ml) and added water (50 ml). Layers were separated and the organic layer was washed with aq. sat. Na₂SO₃ (50 ml) and brine (50 ml) successively. The organic layer was dried over anhydrous sodium sulphate, filtered, and concentrated in vacuo to give 1.8 g of crude compound. This crude residue was purified by combiflash (R_{f}200, Teledyne/Isco) instrument onto a redisep® R_{f} column with gradient elution (0 to 25%) of ethyl acetate in petroleum ether to afford the title compound (1 g, 78%) as an off white solid.

¹H NMR (400 MHz, DMSO-d6) δ 10.46 (d, J=0.8 Hz, 1H), 8.21 (d, J=0.7 Hz, 1H), 7.29-7.22 (m, 4H), 6.90-6.83 (m, 4H), 6.82-6.75 (m, 2H), 4.51 (s, 4H), 3.71 (s, 6H).; LCMS m/z=452.92 (M+; 50%).

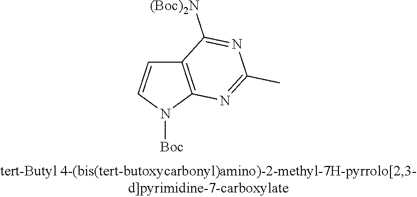

To a solution of commercially available 2-methyl-7H-pyrrolo[2,3-d]pyrimidin-4-amine (0.400 g, 2.70 mmol) in acetonitrile (10 ml) and methylene chloride (10 ml), was added di-tert-butyl dicarbonate (6.27 ml, 27.0 mmol) and DMAP (0.066 g, 0.540 mmol) at 25° C. The resulting mixture was stirred at 25° C. for 16 h. Volatiles were removed in vacuo and the crude residue was purified by combiflash (R_{f}200, Teledyne/Isco) instrument onto a redisep® R_{f}column with gradient elution (0 to 20%) of ethyl acetate in petroleum ether to afford the title compound (0.7 g,57.8%) as an off-white solid. ¹H NMR (400 MHz, Chloroform-d) δ 7.61 (d, J=4.1 Hz, 1H), 6.44 (d, J=4.1 Hz, 1H), 2.86 (s, 3H), 1.70 (s, 9H), 1.43 (s, 18H).; LCMS m/z=448.42 (M+; 50%).

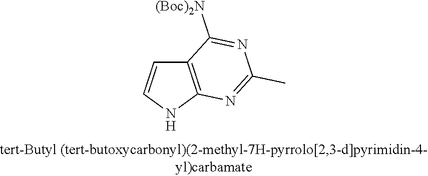

To a solution of tert-butyl 4-(bis(tert-butoxycarbonyl)amino)-2-methyl-7H-pyrrolo[2,3-d]pyrimidine-7-carboxylate (0.7 g, 1.561 mmol) in MeOH (7 ml) was added triethylamine (2.393 ml, 17.17 mmol) at 25° C. The resulting mixture was stirred at 60° C. for 18 h. Volatiles were removed in vacuo and the crude residue was purified by combiflash (R_{f}200, Teledyne/Isco) instrument onto a redisep® R_{f} column with gradient elution (0 to 15%) of ethyl acetate in petroleum ether to afford the title compound (0.4 g, 73.6%) as an off-white solid. ¹H NMR (400 MHz, DMSO-d6) δ 12.05 (s, 1H), 7.49 (dd, J=3.6, 2.2 Hz, 1H), 6.27 (dd, J=3.6, 1.7 Hz, 1H), 2.59 (s, 3H), 1.41 (s, 18H); LCMS m/z=349.41 (M+1; 10%).

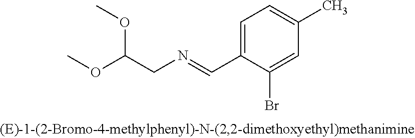

A mixture of 2-bromo-4-methylbenzaldehyde (73 g, 367 mmol) and 2,2-dimethoxyethan-1-amine (47.9 ml, 440 mmol) in toluene (450 ml) was heated to 120° C. with a Dean-Stark apparatus for 4 h. Volatiles were removed in vacuo and this crude compound was carried forward for the next step without further purification. ¹H NMR (400 MHz, DMSO-d6) δ 8.54 (s, 1H), 7.83 (d, J=8.0 Hz, 1H), 7.54 (dd, J=1.7, 0.9 Hz, 1H), 7.29-7.25 (m, 1H), 4.64 (t, J=5.4 Hz, 1H), 3.74 (dd, J=5.4, 1.4 Hz, 2H), 3.30 (s, 6H), 2.34 (s, 3H).

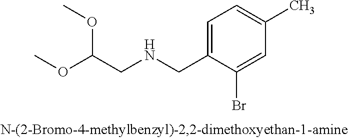

To a solution of (E)-1-(2-bromo-4-methylphenyl)-N-(2,2-dimethoxyethyl)methanimine (105 g, 367 mmol) in ethanol (820 ml), was added sodium borohydride (20.82 g, 550 mmol) portion-wise at 0° C. The resulting mixture was stirred at 25° C. for 2 h. Acetone (100 ml) was slowly added to quench the reaction. Volatiles were removed in vacuo and the crude residue was purified by combiflash (R_{f}200, Teledyne/Isco) instrument onto a redisep® R_{f}column with gradient elution (0 to 2%) of methanol in dichloromethane to afford the title compound (83 g, 78%) as an off-white solid. ¹H NMR (400 MHz, DMSO-d6) δ 7.44-7.40 (m, 1H), 7.36 (d, J=7.7 Hz, 1H), 7.20-7.15 (m, 1H), 4.41 (t, J=5.5 Hz, 1H), 3.72 (s, 2H), 3.25 (s, 6H), 2.58 (d, J=5.5 Hz, 2H), 2.28 (s, 3H); LCMS m/z=288.1 (M+; 100%).

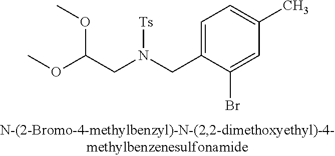

To a stirred solution of N-(2-bromo-4-methylbenzyl)-2,2-dimethoxyethan-1-amine (83 g, 288 mmol) in DCM (1000 ml), was added pyridine (116 ml, 1440 mmol) followed by solution of p-toluenesulfonyl chloride (93 g, 490 mmol) in DCM (300 ml) dropwise at 25° C. The resulting mixture was stirred for 16 h. The reaction mixture was diluted with water (1000 ml) and extracted with methylene chloride (1000 ml). Layers were separated and the organic layer was washed with brine (500 ml). The organic layer was dried over anhydrous sodium sulphate, filtered, and concentrated in vacuo to give 130 g of crude compound. This crude residue was purified by combiflash (R_{f}200, Teledyne/Isco) instrument onto a redisep® R_{f} column with gradient elution (0 to 25%) of ethyl acetate in petroleum ether to afford the title compound (120 g, 94%) as a colourless oil. ¹H NMR (400 MHz, Chloroform-d) δ 7.81-7.71 (m, 2H), 7.42-7.27 (m, 4H), 7.16-7.06 (m, 1H), 4.52 (s, 2H), 4.37 (t, J=5.3 Hz, 1H), 3.31 (d, J=5.4 Hz, 2H), 3.23 (s, 6H), 2.45 (s, 3H), 2.32 (s, 3H); LCMS m/z=507.05; 10%).

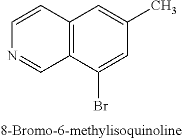

To an ice cooled solution of aluminum chloride (217 g, 1628 mmol) in DCM (1400 ml) was added a solution of N-(2-bromo-4-methylbenzyl)-N-(2,2-dimethoxyethyl)-4-methylbenzenesulfonamide (120 g, 271 mmol) in DCM (600 ml) dropwise at 0° C. The resulting mixture was allowed to warm to 25° C. and stirred at this temperature for 16 h. The reaction mixture was diluted with cold water (2 lit.) and 500 ml of DCM and stirred for 1 h. Layers were separated, and the aqueous layer was again extracted with DCM (500 ml×2). The combined organic layer was washed with brine (500 ml), dried over anhydrous sodium sulphate, filtered, and concentrated in vacuo to give 80 g of crude compound. This crude residue was purified by combiflash (R_{f}200, Teledyne/Isco) instrument onto a redisep® R_{f} column with gradient elution (0 to 50%) of ethyl acetate in petroleum ether to afford the title compound (50 g, 83%) as an off white solid. ¹H NMR (400 MHz, Chloroform-d) δ 9.54 (s, 1H), 8.57 (d, J=5.7 Hz, 1H), 7.71 (d, J=1.5 Hz, 1H), 7.61-7.51 (m, 2H), 2.54 (s, 3H); LCMS m/z=222, 224 (M+, M+2; 100%).

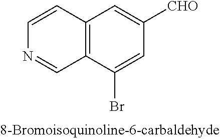 [SMILES: O=Cc1cc(Br)c2cnccc2c1]

A suspension of selenium dioxide (28.0 g, 252 mmol) and 8-bromo-6-methylisoquinoline (20 g, 90 mmol) in 1,2-dichlorobenzene (120 ml) was heated at 180° C. for 7 h. The reaction mixture was diluted with 25% MeOH in DCM (500 ml) and filtered through a celite bed, washed with 25% MeOH in DCM (500 ml). The filtrate was concentrated under reduced pressure and the residue was purified by combiflash (Rf200, Teledyne/Isco) instrument onto a redisep® Rf column with gradient elution (0 to 70%) of ethyl acetate in petroleum ether to afford the title compound (5.1 g, 23.99%) as an off white solid. ¹H NMR (400 MHz, DMSO-d6) δ 10.17 (s, 1H), 9.57 (d, J=1.0 Hz, 1H), 8.80 (d, J=5.6 Hz, 1H), 8.68 (t, J=1.1 Hz, 1H), 8.29 (d, J=1.4 Hz, 1H), 8.15 (dd, J=5.6, 0.9 Hz, 1H); GCMS m/z=235.07, 237.07 (M+, M+2; 100%).

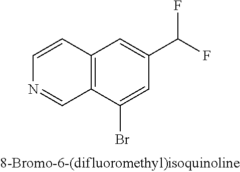 [SMILES: FC(F)c1cc(Br)c2cnccc2c1]

To a stirred solution of 8-bromoisoquinoline-6-carbaldehyde (5 g, 21.18 mmol) in DCM (120 ml) was slowly added DAST (28.0 ml, 212 mmol) at 0° C. The resulting mixture was stirred at 0° C. for 15 mins & then at 25° C. for 16 h. The reaction was diluted with DCM (50 ml) and quenched with cold aqueous saturated NaHCO₃ solution (100 ml). The reaction mixture was stirred at 25° C. until CO₂ evolution ceased (~20 mins.). Layers were separated and the aqueous layer was again extracted with DCM (50 ml×2). The combined organic layer was washed with brine (50 ml), dried over anhydrous sodium sulphate, filtered, and concentrated in vacuo to give 5.1 g of a crude compound. This crude residue was purified by combiflash (Rf200, Teledyne/Isco) instrument onto a redisep® Rf column with gradient elution (0 to 30%) of ethyl acetate in petroleum ether to afford the title compound (3.3 g, 60.4%) as an off white solid. ¹H NMR (400 MHz, Chloroform-d) δ 9.70 (s, 1H), 8.73 (d, J=5.7 Hz, 1H), 8.04-7.96 (m, 2H), 7.75 (d, J=5.8 Hz, 1H), 6.81 (t, J=55.8 Hz, 1H); GCMS m/z=257.04, 259.04 (M+, M+2; 100%).

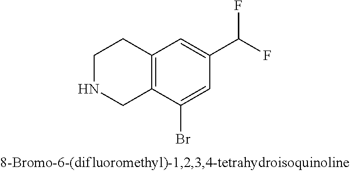 [SMILES: FC(F)c1cc(Br)c2c(c1)CCNC2]

To a stirred solution of 8-bromo-6-(difluoromethyl)isoquinoline (2.22 g, 8.60 mmol) in acetic Acid (50 ml) was added NaBH₄ (1.139 g, 30.1 mmol) in portions at 25° C. The reaction was stirred at 25° C. for 1.5 h. Volatiles were removed in vacuo at 40° C. and the crude residue was diluted with DCM (50 ml) and washed with sat. aq. NaHCO₃ (100 ml). Layers were separated and the organic layer was washed with brine (50 ml). The organic layer was dried over anhydrous sodium sulphate, filtered, and concentrated in vacuo to give 2.2 g of crude compound, which was carried to next step without further purification. ¹H NMR (400 MHz, Chloroform-d) δ 7.54 (s, 1H), 7.22 (s, 1H), 6.58 (t, J=56.3 Hz, 1H), 4.02 (s, 2H), 3.13 (t, J=5.9 Hz, 2H), 2.86 (t, J=5.9 Hz, 2H).

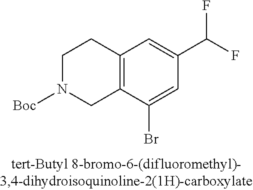 [SMILES: CC(C)(C)OC(=O)N1CCc2cc(C(F)F)cc(Br)c2C1]

To a stirred solution of 8-bromo-6-(difluoromethyl)-1,2,3,4-tetrahydroisoquinoline (5 g, 19.08 mmol) in DCM (45 ml) was added Et₃N (5.32 ml, 38.2 mmol) and di-tert-butyl dicarbonate (5.32 ml, 22.89 mmol) at 0° C. The resulting mixture was stirred at 25° C. for 2 h. The reaction mixture was diluted with water (100 ml) and extracted with DCM (100 ml×2). Layers were separated and the organic layer was washed with brine (100 ml). The organic layer was dried over anhydrous sodium sulphate, filtered, and concentrated in vacuo to give 6.2 g of a crude compound. This crude residue was purified by combiflash (R_{f}200, Teledyne/Isco) instrument onto a redisep® R_{f}column with gradient elution (0 to 7%) of ethyl acetate in petroleum ether to afford the title compound (5.8 g, 84%) as an off white solid. ¹H NMR (400 MHz, Chloroform-d) δ 7.59 (s, 1H), 7.26 (s, 1H), 6.59 (t, J=56.3 Hz, 1H), 4.58 (s, 2H), 3.67 (t, J=5.8 Hz, 2H), 2.90 (t, J=5.9 Hz, 2H), 1.53 (s, 9H).

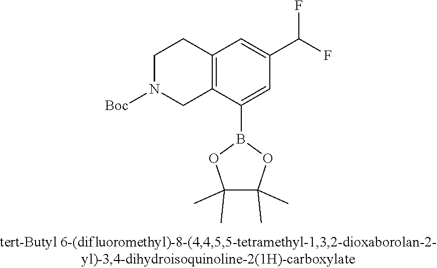 [SMILES: CC(C)(C)OC(=O)N1CCc2cc(C(F)F)cc(B3OC(C)(C)C(C)(C)O3)c2C1]

[1,1′-Bis(diphenylphosphino)ferrocene]dichloropalladium(II) (0.586 g, 0.801 mmol) was added in one portion to a degassed mixture of tert-butyl 8-bromo-6-(difluoromethyl)-3,4-dihydroisoquinoline-2(1H)-carboxylate (5.8 g, 16.01 mmol), Bis(pinacolato)diboron (8.13 g, 32.0 mmol) and potassium acetate (6.29 g, 64.1 mmol) in Dioxane (60 ml) and the reaction mixture was heated to 100° C. for 2 h. The mixture was then cooled to 25° C. and filtered through a celite bed, washed with ethyl acetate (25 ml). The filtrate was concentrated in vacuo to give 6.8 g of a crude compound. This crude residue was purified by combiflash (R_{f}200, Teledyne/Isco) instrument onto a redisep® R_{f} column with gradient elution (0 to 5%) of ethyl acetate in petroleum ether to afford the title compound (6.5 g, 99%) as an off white solid. ¹H NMR (400 MHz, Chloroform-d) δ 7.81 (s, 1H), 7.38 (s, 1H), 6.62 (t, J=56.5 Hz, 1H), 4.91 (s, 2H), 3.66 (t, J=6.0 Hz, 2H), 2.91 (t, J=6.0 Hz, 2H), 1.37 (s, 9H), 1.28 (s, 12H); LCMS m/z=410.23 (M+1; 20%).

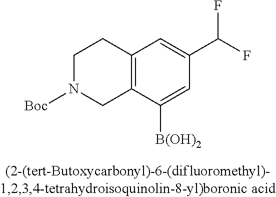 [SMILES: CC(C)(C)OC(=O)N1CCc2cc(C(F)F)cc(B(O)O)c2C1]

Sodium periodate (1.787 g, 8.36 mmol) was added to a mixture of tert-butyl 6-(difluoromethyl)-8-(4,4,5,5-tetramethyl-1,3,2-dioxaborolan-2-yl)-3,4-dihydroisoquinoline-2(1H)-carboxylate (1.14 g, 2.79 mmol) in water (2.5 ml) and acetone (12.50 ml) at 25° C. The resulting mixture was stirred at 25° C. for 1 h. 1N aqueous HCl (2.79 ml, 2.79 mmol) was added and stirred the reaction mixture for an additional 4 h. The reaction mixture was diluted with ethyl acetate (50 ml) and washed with water (50 ml). Layers were separated, the organic layer was washed with brine (50 ml) and dried over anhydrous sodium sulphate. The organic layer was filtered and concentrated in vacuo to give 0.8 g of a crude compound. This crude residue was purified by combiflash (R_{f}200, Teledyne/Isco) instrument onto a redisep® R_{f} column with gradient elution (0 to 10%) of methanol in dichloromethane to afford the title compound (0.61 g, 66.9%) as an off-white solid. ¹H NMR (400 MHz, DMSO-d6) δ 8.29 (s, 2H), 7.56 (s, 1H), 7.35 (s, 1H), 6.97 (t, J=56.0 Hz, 1H), 4.71 (s, 2H), 3.53 (t, J=6.1 Hz, 2H), 2.84 (t, J=6.0 Hz, 2H), 1.42 (s, 9H). LCMS m/z=328.34 (M+1; 10%).

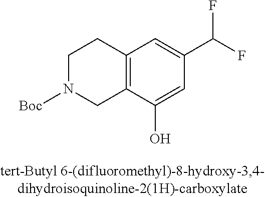 [SMILES: CC(C)(C)OC(=O)N1CCc2cc(C(F)F)cc(O)c2C1]

To a mixture of (2-(tert-butoxycarbonyl)-6-(difluoromethyl)-1,2,3,4-tetrahydroisoquinolin-8-yl)boronic acid (1.5 g, 4.59 mmol) in aq. citric acid solution (36 ml, 0.459 mmol) was added hydrogen peroxide (0.468 ml, 4.59 mmol) at 25° C. and stirred for 3 h. The reaction mixture was diluted with ethyl acetate (50 ml) and washed with water (50 ml). Layers were separated, the organic layer was washed with brine (50 ml) and dried over anhydrous sodium sulphate. The organic layer was filtered and concentrated in vacuo to give 1.2 g of a crude compound. This crude residue was purified by combiflash (R_{f}200, Teledyne/Isco) instrument onto a redisep® R_{f} column with gradient elution (0 to 20%) of methanol in dichloromethane to afford the title compound (0.92 g, 67%) as an off-white solid. ¹H NMR (400 MHz, Chloroform-d) δ 6.82 (s, 1H), 6.77 (s, 1H), 6.53 (t, J=56.6 Hz, 1H), 4.59 (s, 2H), 3.67 (t, J=5.9 Hz, 2H), 2.84 (t, J=5.9 Hz, 2H), 1.53 (s, 9H).

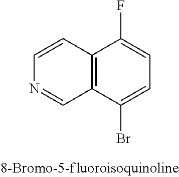 [SMILES: Fc1ccc(Br)c2cnccc12]

The title compound was prepared by following same reaction protocol as described in WO2018/167800 A1.

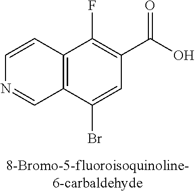 [SMILES: O=C(O)c1cc(Br)c2cnccc2c1F]

To a stirred solution of 8-bromo-5-fluoroisoquinoline (1.7 g, 7.52 mmol) in THF (15 ml) was added LDA (2 M in THF/heptane/ethylbenzene) (5.64 ml, 11.28 mmol) at −78° C. and stirred for 1 h. DMF (1.747 ml, 22.56 mmol) was added at −78° C. and stirred for 30 min. The resulting mixture was quenched with ice water and allowed to warm to room temperature. The reaction mixture was diluted with ethyl acetate (20 ml) and washed with water (20 ml). Layers were separated, and the organic layer was washed with brine (20 ml) and dried over anhydrous Na₂SO₄. The organic layer was filtered and concentrated in vacuo to give 1 g of a crude compound. This residue was purified by combiflash (R_{f}200, Teledyne/Isco) instrument onto a redisep® R_{f} column with gradient elution (0 to 10%) of ethyl acetate in petroleum ether to afford (0.467 g, 24.44%) of the title compound. LCMS m/z=254.14 (M+; 90%).

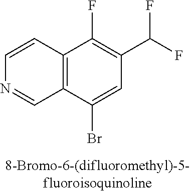 [SMILES: Fc1c(C(F)F)cc(Br)c2cnccc12]

To a stirred solution of 8-bromo-5-fluoroisoquinoline-6-carbaldehyde (233 mg, 0.917 mmol) in DCM (6 ml) was added DAST (0.606 ml, 4.59 mmol) at 0° C. and stirred the reaction mixture for 15 min. The resulting mixture was allowed to warm to room temperature and stirred for 16 h. The resulting mixture was diluted with dichloromethane (10 ml) and quenched with cold saturated aqueous NaHCO₃ solution (20 ml). Stirred the reaction mixture at room temperature for 20 mins. Layers were separated, and the organic layer was washed with brine (20 ml) and dried over anhydrous Na₂SO₄. The organic layer was filtered and concentrated in vacuo to give 0.8 g of a crude compound. This residue was purified by combiflash (R_{f}200, Teledyne/Isco) instrument onto a redisep® R_{f} column with gradient elution (0 to 10%) of ethyl acetate in petroleum ether to afford (0.166 g, 65.6%) of the title compound. LCMS m/z=276.02 (M+; 100%).

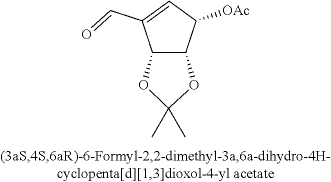 [SMILES: CC(=O)O[C@H]1C=C(C=O)[C@H]2OC(C)(C)O[C@@H]12]

To a stirred solution of (3aS,4S,6aR)-6-(hydroxymethyl)-2,2-dimethyl-3a,6a-dihydro-4H-cyclopenta[d][1,3]dioxol-4-yl acetate, which was synthesized as described in Bio-organic and medicinal Chemistry, 1996, 4(7), 1077-1088, (18 g, 79 mmol) in DCM (500 ml) at 0° C. was added Dess-Martin Periodinane (66.9 g, 158 mmol) portion wise under N₂ atmosphere. The reaction mixture was slowly warmed to 25° C. and stirred for 2 h. The reaction mixture was diluted with dichloromethane (200 ml) and washed with water (200 ml). Layers were separated, the organic layer was washed with brine (100 ml) and dried over anhydrous sodium sulphate. The organic layer was filtered and concentrated in vacuo to give 29 g of a crude compound. This crude residue was purified by combiflash (R_{f}200, Teledyne/Isco) instrument onto a redisep® R_{f} column with gradient elution (0 to 35%) of ethyl acetate in petroleum ether to afford the title compound (15.5 g, 87%) as a pale yellow liquid. ¹H NMR (400 MHz, Chloroform-d) δ 9.92 (s, 1H), 6.78 (d, J=2.1 Hz, 1H), 5.55 (dd, J=5.6, 2.1 Hz, 1H), 5.28 (d, J=5.6 Hz, 1H), 5.02 (t, J=5.6 Hz, 1H), 2.19 (s, 3H), 1.41 (s, 6H).

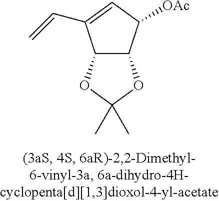 [SMILES: C=CC1=C[C@H](OC(C)=O)[C@@H]2OC(C)(C)O[C@H]12]

To a stirred solution of methyltriphenylphosphonium bromide (6.16 g, 17.24 mmol) in dry THF (50 ml) was added n-BuLi (2.5M in hexane; 6.37 ml, 15.91 mmol) dropwise at −15° C. and stirred for 30 min at same temperature. The solution of (3aS,4S,6aR)-6-formyl-2,2-dimethyl-3a,6a-dihydro-4H-cyclopenta[d][1,3]dioxol-4-yl acetate (3.0 g, 13.26 mmol) in dry THF (15 ml) was added dropwise in above reaction mixture at −15° C. and slowly allowed to attain 0° C. to 10° C. within 1 h. The reaction mixture quenched with saturated solution of ammonium chloride (50 mL) diluted with in ethyl acetate (70 mL). Layers were separated, the combined organic layer was washed with brine (30 mL), dried over anhydrous Na₂SO₄, filtered and concentrated in vacuo to give 4.5 g of a crude compound. This residue was purified by combiflash (R_{f}200, Teledyne/Isco) instrument onto a redisep® R_{f} column with gradient elution (4-6%) of ethyl acetate in petroleum ether to afford the title compound as a colourless liquid. ¹H NMR (400 MHz, Chloroform-d) δ 6.49 (dd, J=17.7, 10.8 Hz, 1H), 5.76-5.63 (m, 2H), 5.45-5.35 (m, 2H), 5.18 (d, J=5.8 Hz, 1H), 4.93 (t, J=5.8 Hz, 1H), 2.14 (s, 3H), 1.44-1.39 (m, 6H).

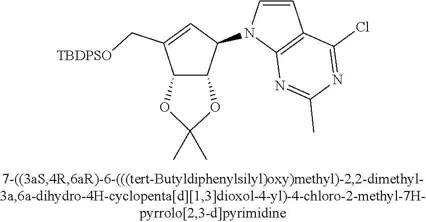

To a stirred solution of (3aS,4S,6aR)-6-(((tert-butyldiphenylsilyl)oxy)methyl)-2,2-dimethyl-3a,6a-dihydro-4H-cyclopenta[d][1,3]dioxol-4-ol (0.1 g, 0.236 mmol), which was synthesized as described in Kenneth A. Jacobson et.al; Purinergic Signaling (2015) 11:371-387, in THF (5 ml) was added commercially available 4-chloro-2-methyl-7H-pyrrolo[2,3-d]pyrimidine (0.079 g, 0.471 mmol), triphenylphosphine (0.247 g, 0.942 mmol) at 0° C. DIAD (0.183 ml, 0.942 mmol) was added and stirred for 10 min. Warmed the reaction to 25° C. and stirred for 12 h. Volatiles were removed in vacuo and the crude residue was purified by combiflash (R_{f}200, Teledyne/Isco) instrument onto a redisep® R_{f}column with gradient elution (0 to 10%) of ethyl acetate in petroleum ether to afford the title compound (0.1 g, 73.9%) as a colourless oil. ¹H NMR (400 MHz, DMSO-d6) δ 7.65 (ddd, J=8.1, 6.7, 1.5 Hz, 4H), 7.50-7.39 (m, 6H), 7.33 (d, J=3.7 Hz, 1H), 6.65 (d, J=3.6 Hz, 1H), 5.77 (s, 2H), 5.41 (d, J=5.6 Hz, 1H), 4.65 (d, J=5.6 Hz, 1H), 4.51 (d, J=15.8 Hz, 1H), 4.39 (d, J=15.8 Hz, 1H), 2.64 (s, 3H), 1.34 (s, 3H), 1.25 (s, 3H), 1.02 (s, 9H); LCMS m/z=574.08 (M+; 100%).

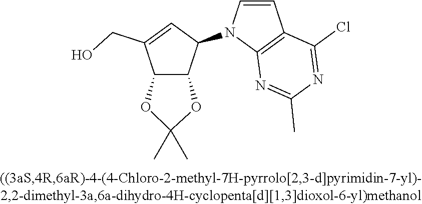 [SMILES: Cc1nc(Cl)c2ccn([C@@H]3C=C(CO)[C@H]4OC(C)(C)O[C@H]43)c2n1]

To a stirred solution of 7-((3aS,4R,6aR)-6-(((tert-butyldiphenylsilyl)oxy)methyl)-2,2-dimethyl-3a,6a-dihydro-4H-cyclopenta[d][1,3]dioxol-4-yl)-4-chloro-2-methyl-7H-pyrrolo[2,3-d]pyrimidine (2.1 g, 3.66 mmol) in THF (20 ml) was added TBAF (5.12 ml, 5.12 mmol) slowly and stirred the reaction mixture at 25° C. for 15 h. Volatiles were removed in vacuo and the crude residue was purified by combiflash (R_{f}200, Teledyne/Isco) instrument onto a redisep® R_{f}column with gradient elution (0 to 30%) of ethyl acetate in petroleum ether to afford the title compound (1 g, 81%) as a colourless oil. ¹H NMR (400 MHz, DMSO-d6) δ 7.41 (d, J=3.7 Hz, 1H), 6.63 (d, J=3.6 Hz, 1H), 5.75-5.70 (m, 1H), 5.65 (d, J=2.4 Hz, 1H), 5.37 (d, J=5.7 Hz, 1H), 5.09 (t, J=5.6 Hz, 1H), 4.58 (d, J=5.7 Hz, 1H), 4.17 (dd, J=5.1, 2.5 Hz, 2H), 2.67 (s, 3H), 1.40 (s, 3H), 1.28 (s, 3H); LCMS m/z=336.28 (M+1; 30%).

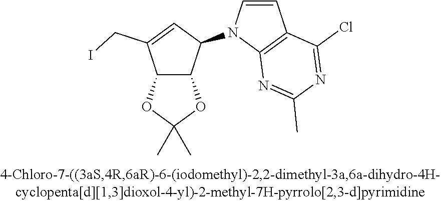 [SMILES: Cc1nc(Cl)c2ccn([C@@H]3C=C(CI)[C@H]4OC(C)(C)O[C@H]43)c2n1]

To a stirred solution of imidazole (0.223 g, 3.28 mmol) and triphenylphosphine (0.508 g, 1.936 mmol) in DCM (10 ml) was added iodine (0.491 g, 1.936 mmol) slowly at 0° C. A solution of ((3aS,4R,6aR)-4-(4-chloro-2-methyl-7H-pyrrolo[2,3-d]pyrimidin-7-yl)-2,2-dimethyl-3a,6a-dihydro-4H-cyclopenta[d][1,3]dioxol-6-yl)methanol (0.5 g, 1.489 mmol) dissolved in DCM (10 ml) was added thereto and stirred for 10 min. The resulting mixture was stirred for 3 h at 25° C. The reaction mixture was diluted with dichloromethane (50 ml) and washed with water (50 ml). Layers were separated, the organic layer was washed with brine (50 ml) and dried over anhydrous sodium sulphate. The organic layer was filtered and concentrated in vacuo to give 0.6 g of a crude compound. This crude residue was purified by combiflash (R_{f}200, Teledyne/Isco) instrument onto a redisep® R_{f} column with gradient elution (0 to 6%) of ethyl acetate in petroleum ether to afford the title compound (0.34 g, 51.2%) as a light brown oil. LCMS m/z=446 (M+; 100%).

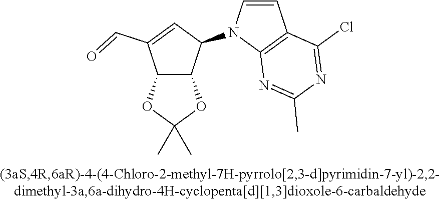 [SMILES: Cc1nc(Cl)c2ccn([C@@H]3C=C(C=O)[C@H]4OC(C)(C)O[C@H]43)c2n1]

To a cold solution (0° C.) of ((3aS,4R,6aR)-4-(4-chloro-2-methyl-7H-pyrrolo[2,3-d]pyrimidin-7-yl)-2,2-dimethyl-3a,6a-dihydro-4H-cyclopenta[d][1,3]dioxol-6-yl)methanol (0.9 g, 2.68 mmol) in DCM (50 ml) was added Dess-Martin Periodinane (5.68 g, 13.40 mmol) portion-wise and stirred the reaction mixture for 10 min. The resulting mixture was stirred for 4 h at 25° C.

Volatiles were removed in vacuo and the crude residue was purified by combiflash (R_{f}200, Teledyne/Isco) instrument onto a redisep® R_{f}column with gradient elution (0 to 20%) of ethyl acetate in petroleum ether to afford the title compound (0.75 g, 84%) as a colourless oil. ¹H NMR (400 MHz, DMSO-d6) δ 9.90 (s, 1H), 7.58 (d, J=3.7 Hz, 1H), 7.05 (dd, J=2.6, 0.9 Hz, 1H), 6.66 (d, J=3.7 Hz, 1H), 6.0-5.96 (m, 1H), 5.64 (dd, J=5.9, 1.5 Hz, 1H), 4.81-4.77 (m, 1H), 2.65 (s, 3H), 1.40 (s, 3H), 1.29 (s, 3H); LCMS m/z=334.34 (M+1; 100%).

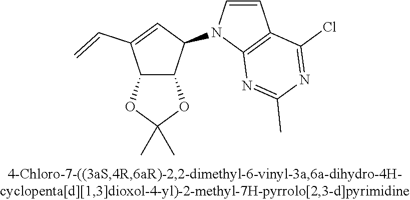 [SMILES: C=CC1=C[C@@H](n2ccc3c(Cl)nc(C)nc32)[C@@H]2OC(C)(C)O[C@H]12]

To a suspension of methyltriphenyl phosphonium bromide (0.783 g, 2.193 mmol) in THF (6 ml) was added KHMDS (2.193 ml, 2.193 mmol) slowly at 0° C. After 5 min, a solution of (3aS,4R,6aR)-4-(4-chloro-2-methyl-7H-pyrrolo[2,3-d]pyrimidin-7-yl)-2,2-dimethyl-3a,6a-dihydro-4H-cyclopenta[d][1,3]dioxole-6-carbaldehyde (0.61 g, 1.828 mmol) in THF (3 ml) was added slowly and stirred the reaction at same temperature for 5 min. The reaction mixture was diluted with ethyl acetate (5 ml) and washed with water (2 ml). Layers were separated, the organic layer was washed with brine (5 ml) and dried over anhydrous sodium sulphate. The organic layer was filtered and concentrated in vacuo to give 0.36 g of a crude compound. This crude residue was purified by combiflash (R_{f}200, Teledyne/Isco) instrument onto a redisep® R_{f} column with gradient elution (0 to 10%) of ethyl acetate in petroleum ether to afford the title compound (0.28 g, 46.2%) as an off white solid. ¹H NMR (400 MHz, DMSO-d6) δ 7.43 (d, J=3.7 Hz, 1H), 6.65-6.55 (m, 2H), 5.85 (d, J=2.7 Hz, 1H), 5.79 (s, 1H), 5.64 (d, J=1.7 Hz, 1H), 5.63-5.59 (m, 1H), 5.42 (dd, J=10.8, 1.7 Hz, 1H), 4.68 (dd, J=5.9, 1.0 Hz, 1H), 2.66 (s, 3H), 1.39 (s, 3H), 1.32 (s, 3H); LCMS m/z=332.11 (M+1; 100%).

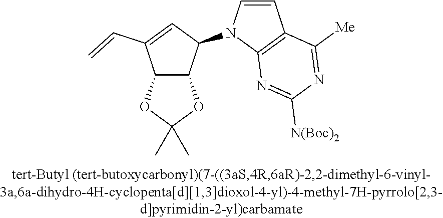

To a solution of (3aS,4S,6aR)-2,2-dimethyl-6-vinyl-3a,6a-dihydro-4H-cyclopenta[d][1,3]dioxol-4-ol (1 g, 5.49 mmol), which was synthesized as described in Organic letters, 2012, 14, 2134-2137, triphenylphosphine (3.6 g, 13.72 mmol), tert-butyl (tert-butoxycarbonyl)(4-methyl-7H-pyrrolo[2,3-d]pyrimidin-2-yl)carbamate (2.103 g, 6.04 mmol), which was synthesized as described in US2016/244475 A1, at 0° C. was added DIAD (2.67 ml, 13.72 mmol) slowly. After stirring for 10 mins, the reaction mixture was stirred at 25° C. for 3 h. Volatiles were removed in vacuo and the crude residue was purified by combiflash (R_{f}200, Teledyne/Isco) instrument onto a redisep® R_{f}column with gradient elution (0 to 30%) of ethyl acetate in petroleum ether to afford 2 g of sufficiently pure desired compound. This residue was then purified by reverse phase preparative HPLC (YMC Triart C18 250×50 mm,10 μm) with gradient elution of mobile phase B in mobile phase A (40:60 mobile phase A: mobile phase B to 20:80 mobile phase A: mobile phase B; Mobile Phase A: Water:CH₃CN (95:5, V/V)+0.1% NH₄OH, Mobile Phase B: CH₃CN:Water (90:10,V/V)+0.1% NH₄OH; Wavelength: 220 nm; Flow Rate: 117 ml/min; Run time: 20 min) to afford (0.61 g, 21.68%) of the title compound as an off white solid. ¹H NMR (400 MHz, DMSO-d6) δ 7.39 (d, J=3.6 Hz, 1H), 6.76 (d, J=3.6 Hz, 1H), 6.61 (dd, J=17.6, 10.8 Hz, 1H), 5.89 (d, J=2.6 Hz, 1H), 5.71 (s, 1H), 5.65-5.55 (m, 2H), 5.45-5.37 (m, 1H), 4.59 (d, J=5.9 Hz, 1H), 2.66 (s, 3H), 1.40 (s, 18H), 1.37 (s, 3H), 1.30 (s, 3H); LCMS m/z=512.44 (M+; 50%).

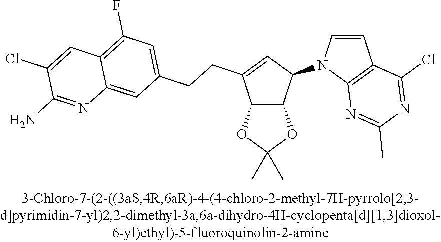 [SMILES: Cc1nc(Cl)c2ccn([C@@H]3C=C(CCc4cc(F)c5cc(Cl)c(N)nc5c4)[C@H]4OC(C)(C)O[C@H]43)c2n1]

4-Chloro-7-((3aS,4R,6aR)-2,2-dimethyl-6-vinyl-3a,6a-dihydro-4H-cyclopenta[d][1,3]dioxol-4-yl)-2-methyl-7H-pyrrolo[2,3-d]pyrimidine (0.28 g, 0.844 mmol) in 9-BBN (6.75 ml, 3.38 mmol) was heated at 70° C. for 2 h under N₂ atmosphere. The reaction mixture was cooled to 25° C., then potassium phosphate tribasic (0.896 g, 4.22 mmol) in water (1 ml) was added and stirred for 30 mins. A solution of 7-bromo-3-chloro-5-fluoroquinolin-2-amine (0.256 g, 0.928 mmol) in THF (5 ml) was added, followed by dichloro[1,1′-bis(di-t-butylphosphino)ferrocene]palladium(II) (0.055 g, 0.084 mmol). The resulting mixture was stirred at 70° C. for 3 h. The reaction mixture was diluted with ethyl acetate (10 ml) and washed with water (10 ml). Layers were separated, and the organic layer was washed with brine (10 ml) and dried over anhydrous Na₂SO₄. The organic layer was filtered and concentrated in vacuo to give 0.25 g of a crude compound. This residue was purified by combiflash (R_{f}200, Teledyne/Isco) instrument onto a redisep® R_{f} column with gradient elution (0 to 20%) of ethyl acetate in petroleum ether to afford the title compound (0.17 g, 38.1%) as an off-white solid. ¹H NMR (400 MHz, DMSO-d6) δ 8.18 (s, 1H), 7.25 (s, 1H), 7.04 (dd, J=11.0, 1.4 Hz, 1H), 6.97 (s, 2H), 6.92 (d, J=3.7 Hz, 1H), 6.36 (d, J=3.7 Hz, 1H), 5.64 (d, J=3.6 Hz, 1H), 5.49 (s, 1H), 5.38 (d, J=5.6 Hz, 1H), 4.49 (d, J=5.6 Hz, 1H), 3.08-2.94 (m, 2H), 2.71-2.65 (m, 2H), 2.64 (s, 3H), 1.38 (s, 3H), 1.29 (s, 3H); LCMS m/z=528.1 (M+; 100%).

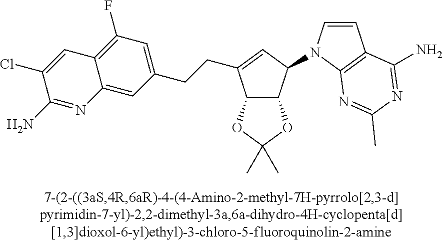 [SMILES: Cc1nc(N)c2ccn([C@@H]3C=C(CCc4cc(F)c5cc(Cl)c(N)nc5c4)[C@H]4OC(C)(C)O[C@H]43)c2n1]

To a mixture of 3-chloro-7-(2-((3aS,4R,6aR)-4-(4-chloro-2-methyl-7H-pyrrolo[2,3-d]pyrimidin-7-yl)-2,2-dimethyl-3a,6a-dihydro-4H-cyclopenta[d][1,3]dioxol-6-yl)ethyl)-5-fluoroquinolin-2-amine (0.170 g, 0.322 mmol) in dioxane (1 ml), was added aq. ammonia (2.089 ml, 97 mmol) at 25° C. and stirred the reaction mixture at 120° C. for 16 h in a steel bomb. Volatiles were removed in vacuo and the crude residue was purified by combiflash (R_{f}200, Teledyne/Isco) instrument onto a redisep® R_{f}column with gradient elution (0 to 2%) of methanol in dichloromethane to afford the title compound (0.08 g, 48.9%) as a colourless oil. LCMS m/z=509.2 (M+1; 25%).

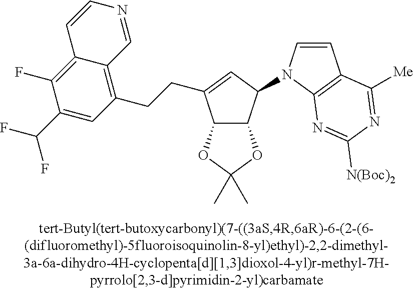

tert-Butyl (tert-butoxycarbonyl)(7-((3aS,4R,6aR)-2,2-dimethyl-6-vinyl-3a,6a-dihydro-4H-cyclopenta[d][1,3]dioxol-4-yl)-4-methyl-7H-pyrrolo[2,3-d]pyrimidin-2-yl)carbamate (0.560 g, 1.092 mmol) in 9-BBN (8.74 ml, 4.37 mmol) was heated at 65° C. for 1.5 h under N₂ atmosphere. The reaction mixture was cooled to 25° C., then potassium phosphate tribasic (0.951 g, 5.46 mmol) in water (2 ml) was added and stirred for 30 mins. A solution of 8-bromo-6-(difluoromethyl)-5-fluoroisoquinoline (0.302 g, 1.092 mmol) in THF (7 ml) was added, followed by the addition of PdCl₂(dppf) (0.080 g, 0.109 mmol). The resulting mixture was stirred at 65° C. for 4 h. The reaction mixture was diluted with ethyl acetate (15 ml) and washed with water (15 ml). Layers were separated, and the organic layer was washed with brine (10 ml) and dried over anhydrous Na₂SO₄. The organic layer was filtered and concentrated in vacuo to give 0.65 g of crude compound. This residue was purified by combiflash (R_{f}200, Teledyne/Isco) instrument onto a redisep® R_{f} column with gradient elution (0 to 20%) of ethyl acetate in petroleum ether to afford the title compound (0.35 g, 45.1%) as an off-white solid. LCMS m/z=710.52 (M+1; 100%).

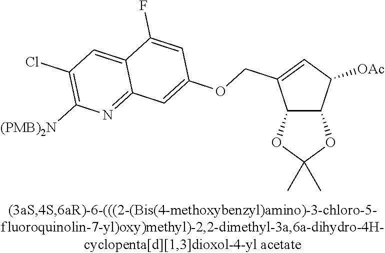

To a cooled solution (0° C.) of (3aS,4S,6aR)-6-(hydroxymethyl)-2,2-dimethyl-3a,6a-dihydro-4H-cyclopenta[d][1,3]dioxol-4-yl acetate, which was synthesized as described in Bio-organic and medicinal Chemistry, 1996, 4(7), 1077-1088, (2 g, 8.76 mmol) in THF (30 ml) was added 2-(bis(4-methoxybenzyl)amino)-3-chloro-5-fluoroquinolin-7-ol (3.97 g, 8.76 mmol), triphenylphosphine (6.89 g, 26.3 mmol) and DIAD (5.11 ml, 26.3 mmol) slowly. After stirring for 30 mins, the reaction mixture was stirred at 25° C. for 15 h. Volatiles were removed in vacuo and the crude residue was purified by combiflash (R_{f}200, Teledyne/Isco) instrument onto a redisep® R_{f}column with gradient elution (0 to 30%) of ethyl acetate in petroleum ether to afford the title compound (3.8 g, 65.4%) as an orange liquid. ¹H NMR (400 MHz, DMSO-d6) δ 8.28 (d, J=0.7 Hz, 1H), 7.28-7.22 (m, 4H), 7.09-6.97 (m, 2H), 6.89-6.83 (m, 4H), 5.88-5.84 (m, 1H), 5.36-5.30 (m, 1H), 5.04 (d, J=5.7 Hz, 1H), 4.91-4.86 (m, 3H), 4.55 (s, 4H), 3.71 (s, 6H), 2.03 (s, 3H), 1.32 (s, 3H), 1.30 (s, 3H); LCMS m/z=663.10 (M+; 100%).

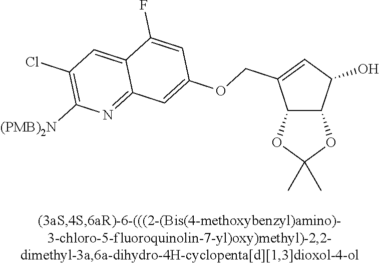

To a stirred suspension of (3aS,4S,6aR)-6-(((2-(bis(4-methoxybenzyl)amino)-3-chloro-5-fluoroquinolin-7-yl)oxy)methyl)-2,2-dimethyl-3a,6a-dihydro-4H-cyclopenta[d][1,3]dioxol-4-yl acetate (3.8 g, 5.73 mmol) in methanol (40 ml) was added K₂CO₃ (1.188 g, 8.60 mmol) at 25° C. The resulting mixture was stirred at 25° C. for 1 h under N₂ atmosphere. Volatiles were removed in vacuo and the crude residue was purified by combiflash (R_{f}200, Teledyne/Isco) instrument onto a redisep® R_{f}column with gradient elution (0 to 50%) of ethyl acetate in petroleum ether to afford the title compound (2.6 g, 73.1%) as an off white solid. LCMS m/z=620.96 (M+; 100%).

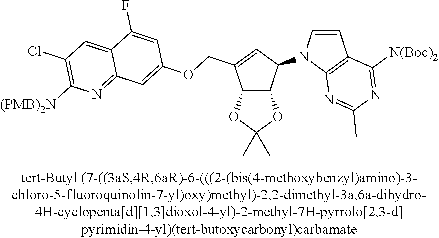

To a stirred solution of (3aS,4S,6aR)-6-(((2-(bis(4-methoxybenzyl)amino)-3-chloro-5-fluoroquinolin-7-yl)oxy)methyl)-2,2-dimethyl-3a,6a-dihydro-4H-cyclopenta[d][1,3]dioxol-4-ol (0.35 g, 0.564 mmol) in THF (15 ml) was added tert-Butyl (tert-butoxycarbonyl)(2-methyl-7H-pyrrolo[2,3-d]pyrimidin-4-yl)carbamate (0.393 g, 1.127 mmol), triphenylphosphine (0.443 g, 1.691 mmol) at 0° C. DEAD (0.268 ml, 1.691 mmol) was added slowly and stirred for 30 min. The resulting mixture was stirred at 25° C. for 16 h. Volatiles were removed in vacuo and the crude residue was purified by combiflash (R_{f}200, Teledyne/Isco) instrument onto a redisep® R_{f}column with gradient elution (0 to 50%) of ethyl acetate in petroleum ether to afford the title compound (0.2 g, 37.3%) as an off white solid. ¹H NMR (400 MHz, DMSO-d₆) δ 8.29 (s, 1H), 7.27-7.24 (m, 4H), 7.10 (d, J=2.3 Hz, 1H), 7.04 (dd, J=11.3, 2.2 Hz, 1H), 6.90-6.82 (m, 4H), 6.27 (d, J=3.7 Hz, 1H), 5.85 (s, 1H), 5.78 (s, 1H), 5.52 (d, J=5.6 Hz, 1H), 5.07-4.91 (m, 3H), 4.65 (d, J=5.7 Hz, 1H), 4.55 (s, 4H), 3.71 (s, 6H), 2.00 (s, 3H), 1.44 (s, 3H), 1.41 (s, 18H), 1.31 (s, 3H); LCMS m/z=951.20 (M+; 100%).

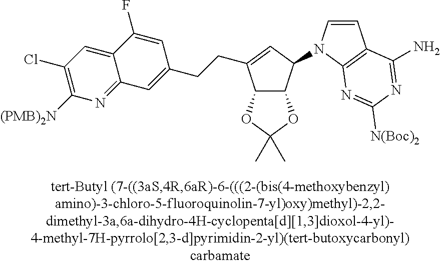

To a stirred solution of (3aS,4S,6aR)-6-(((2-(bis(4-methoxybenzyl)amino)-3-chloro-5-fluoroquinolin-7-yl)oxy)methyl)-2,2-dimethyl-3a,6a-dihydro-4H-cyclopenta[d][1,3]dioxol-4-ol (0.400 g, 0.644 mmol) in THF (15 ml), was added tert-butyl (tert-butoxycarbonyl)(4-methyl-7H-pyrrolo[2,3-d]pyrimidin-2-yl)carbamate (0.449 g, 1.288 mmol), which was synthesized as described in US 2016/244475 A1, triphenylphosphine (0.507 g, 1.932 mmol) at 0° C. DEAD (0.306 ml, 1.932 mmol) was then added slowly and stirred for 30 min. The resulting mixture was stirred at 25° C. for 16 h. Volatiles were removed in vacuo and the crude residue was purified by combiflash (R_{f}200, Teledyne/Isco) instrument onto a redisep® R_{f}column with gradient elution (0 to 18%) of ethyl acetate in petroleum ether to afford the title compound (0.48 g, 78%) as an off white solid. ¹H NMR (400 MHz, Chloroform-d) δ 8.19 (s, 1H), 7.28-7.24 (m, 4H), 7.05-7.00 (m, 2H), 6.91-6.83 (m, 4H), 6.77 (dd, J=11.0, 2.3 Hz, 1H), 6.50 (d, J=3.6 Hz, 1H), 5.92 (s, 1H), 5.84 (d, J=2.6 Hz, 1H), 5.46 (d, J=5.6 Hz, 1H), 4.96-4.88 (m, 2H), 4.65 (d, J=5.6 Hz, 1H), 4.62 (s, 4H), 3.81 (s, 6H), 2.72 (s, 3H), 1.52 (s, 3H), 1.46 (s, 18H), 1.36 (s, 3H); LCMS m/z=951.20 (M+; 100%).

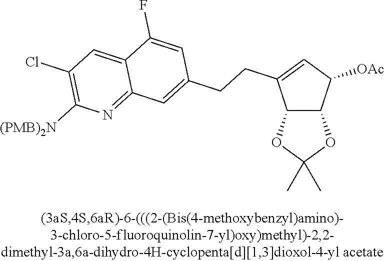

To a stirred solution of (3aS,4S,6aR)-2,2-dimethyl-6-vinyl-3a,6a-dihydro-4H-cyclopenta[d][1,3]dioxol-4-yl acetate (2.000 g, 8.92 mmol) in THF (20 ml) was added 0.5 M 9-BBN (71.3 ml, 35.7 mmol) at 25° C. The resulting mixture was heated at 75° C. for 1.5 h. The reaction mixture was cooled to 25° C., then potassium phosphate tribasic (9.47 g, 44.6 mmol) in water (15 ml) was added and stirred for 20 mins. 7-Bromo-3-chloro-5-fluoro-N,N-bis(4-methoxybenzyl)quinolin-2-amine (3.68 g, 7.13 mmol) and [1,1′-Bis(di-tert-butylphosphino)ferrocene]dichloropalladium(II) (0.291 g, 0.446 mmol) was added at 25° C. and stirred the resulting mixture for 2 h. The reaction mixture was quenched with water (100 ml) and extracted with ethyl acetate (300 ml). Layers were separated, and the organic layer was washed with brine (100 ml) and dried over anhydrous Na₂SO₄. The organic layer was filtered and concentrated in vacuo to give 0.25 g of a crude compound. This residue was purified by combiflash (Rf200, Teledyne/Isco) instrument onto a redisep® Rf column with gradient elution (0 to 30%) of ethyl acetate in petroleum ether to afford the title compound (1.7 g, 28.8%) as a colourless semisolid solid. ¹H NMR (400 MHz, Chloroform-d) δ 8.25 (d, J=0.8 Hz, 1H), 7.42 (s, 1H), 7.29 (d, J=8.6 Hz, 4H), 6.91 (dd, J=10.5, 1.5 Hz, 1H), 6.86 (d, J=8.6, Hz, 4H), 5.55 (q, J=1.7 Hz, 1H), 5.39-5.34 (m, 1H), 4.96-4.88 (m, 2H), 4.60 (s, 4H), 3.81 (s, 6H), 3.05-2.95 (m, 2H), 2.74-2.54 (m, 2H), 2.14 (s, 3H), 1.42 (d, J=7.2 Hz, 6H). LCMS m/z=661.10 (M+; 100%); LCMS m/z=661.22 (M+; 30%).

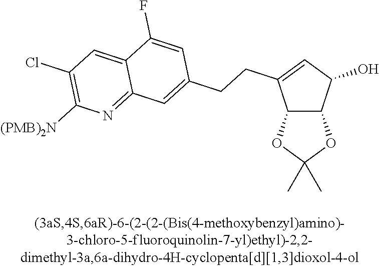

To a stirred solution of (3aS,4S,6aR)-6-(2-(2-(bis(4-methoxybenzyl)amino)-3-chloro-5-fluoroquinolin-7-yl)ethyl)-2,2-dimethyl-3a,6a-dihydro-4H-cyclopenta[d][1,3]dioxol-4-yl acetate (3.4 g, 5.14 mmol) in methanol (60 ml) was added K₂CO₃ (1.066 g, 7.71 mmol) at 25° C. The resulting mixture was stirred at 25° C. for 1 h under N₂ atmosphere. The reaction mixture quenched with water (100 mL) and extracted with ethyl acetate (300 mL). Layers were separated, the combined organic layer was washed with brine (100 mL), dried over anhydrous Na2SO4, filtered and concentrated in vacuo to give 4.1 g of a crude compound. This residue was purified by combiflash (R_{f}200, Teledyne/Isco) instrument onto a redisep® R_{f}column with gradient elution (0-35%) of ethyl acetate in petroleum ether to afford the title compound (2.75 g, 86%) as a colourless semisolid. ¹H NMR (400 MHz, Chloroform-d) δ 8.24 (d, J=0.8 Hz, 1H), 7.42 (s, 1H), 7.32-7.27 (m, 4H), 6.91 (dd, J=10.5, 1.5 Hz, 1H), 6.88-6.83 (m, 4H), 5.61-5.52 (m, 1H), 4.90 (d, J=5.6 Hz, 1H), 4.60 (s, 4H), 4.53 (t, J=8.1 Hz, 1H), 3.81 (s, 6H), 3.04-2.91 (m, 2H), 2.64-2.47 (m, 2H), 1.44 (d, J=3.8 Hz, 6H); LCMS m/z=619.09 (M+; 100%).

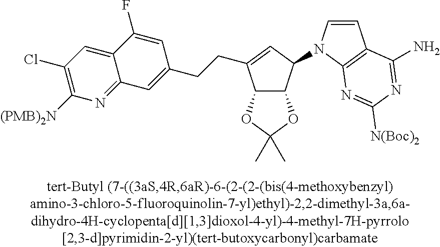

To a stirred solution of (3aS,4S,6aR)-6-(2-(2-(Bis(4-methoxybenzyl)amino)-3-chloro-5-fluoroquinolin-7-yl)ethyl)-2,2-dimethyl-3a,6a-dihydro-4H-cyclopenta[d][1,3]dioxol-4-ol (2.70 g, 4.36 mmol) in THF (50 ml) was added tert-butyl (tert-butoxycarbonyl)(4-methyl-7H-pyrrolo[2,3-d]pyrimidin-2-yl)carbamate (2.279 g, 6.54 mmol), which was synthesized as described in US2016/244475 A1, triphenylphosphine (3.43 g, 13.08 mmol) at 0° C. DEAD (2.071 ml, 13.08 mmol) was then added slowly and stirred for 30 min. The resulting mixture was stirred at 25° C. for 48 h. The reaction mixture was diluted with ethyl acetate 300 ml) and washed with water (100 ml). Layers were separated, and the organic layer was washed with brine (10 ml) and dried over anhydrous Na₂SO₄. The organic layer was filtered and concentrated in vacuo to give 11 g of a crude compound. This residue was purified by reverse phase preparative HPLC to afford the title compound (1.15 g, 27.8%) as an off-white solid. ¹H NMR (400 MHz, Chloroform-d) δ 8.28 (d, J=0.8 Hz, 1H), 7.50 (s, 1H), 7.34-7.24 (m, 6H), 6.96 (dd, J=10.5, 1.4 Hz, 1H), 6.87-6.80 (m, 4H), 6.68 (d, J=3.6 Hz, 1H), 6.34 (d, J=3.6 Hz, 1H), 5.74 (s, 1H), 5.54-5.47 (m, 1H), 5.30 (d, J=5.6 Hz, 1H), 4.63 (s, 4H), 4.51 (d, J=5.6 Hz, 1H), 3.78 (s, 6H), 3.17-3.02 (m, 2H), 2.73 (s, 3H), 1.48 (s, 3H), 1.46 (s, 18H), 1.35 (s, 3H).; LCMS m/z=949.20 (M+; 20%).

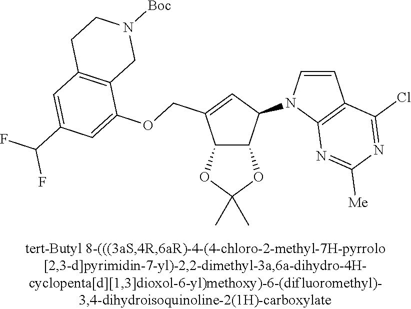 [SMILES: Cc1nc(Cl)c2ccn([C@@H]3C=C(COc4cc(C(F)F)cc5c4CN(C(=O)OC(C)(C)C)CC5)[C@H]4OC(C)(C)O[C@H]43)c2n1]

To a solution of tert-butyl 6-(difluoromethyl)-8-hydroxy-3,4-dihydroisoquinoline-2(1H)-carboxylate (0.054 g, 0.179 mmol) in DMF (2 ml) was added Cs₂CO₃ (0.066 g, 0.202 mmol) at 25° C. and stirred for 30 min. 4-Chloro-7-((3aS,4R,6aR)-6-(iodomethyl)-2,2-dimethyl-3a,6a-dihydro-4H-cyclopenta[d][1,3]dioxol-4-yl)-2-methyl-7H-pyrrolo[2,3-d]pyrimidine (0.100 g, 0.224 mmol) dissolved in DMF (2 ml) was slowly at 0° C. and stirred for 3 h. The reaction mixture was diluted with ethyl acetate (25 ml) and washed with water (25 ml). Layers were separated, and the organic layer was washed with brine (10 ml) and dried over anhydrous Na₂SO₄. The organic layer was filtered and concentrated in vacuo to give 0.11 g of crude compound. This residue was purified by combiflash (R_{f}200, Teledyne/Isco) instrument onto a redisep® R_{f} column with gradient elution (0-30%) of ethyl acetate in petroleum ether to afford the title compound (0.1 g, 72.2%) as an off-white solid. ¹H NMR (400 MHz, Chloroform-d) δ 7.03 (s, 1H), 6.91 (d, J=5.5 Hz, 2H), 6.73-6.43 (m, 2H), 5.87 (s, 2H), 5.50 (d, J=5.5 Hz, 1H), 4.97-4.85 (m, 2H), 4.73 (d, J=5.7 Hz, 1H), 4.59 (s, 2H), 3.65 (s, 2H), 2.89-2.81 (m, 5H), 1.54 (s, 3H), 1.48 (s, 9H), 1.40 (s, 3H); LCMS m/z=616.96 (M+; 10%).

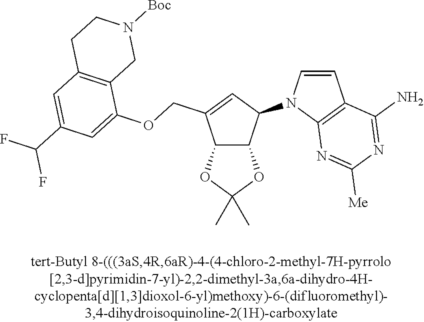 [SMILES: Cc1nc(N)c2ccn([C@@H]3C=C(COc4cc(C(F)F)cc5c4CN(C(=O)OC(C)(C)C)CC5)[C@H]4OC(C)(C)O[C@H]43)c2n1]

To a mixture of tert-butyl 8-(((3aS,4R,6aR)-4-(4-chloro-2-methyl-7H-pyrrolo[2,3-d]pyrimidin-7-yl)-2,2-dimethyl-3a,6a-dihydro-4H-cyclopenta[d][1,3]dioxol-6-yl)methoxy)-6-(difluoromethyl)-3,4-dihydroisoquinoline-2(1H)-carboxylate (0.100 g, 0.162 mmol) in dioxane (1 ml) was added aq. ammonia (0.701 ml, 32.4 mmol) at 25° C. and stirred the reaction mixture at 120° C. for 16 h in a steel bomb. Volatiles were removed in vacuo and the crude residue was purified by combiflash (R_{f}200, Teledyne/Isco) instrument onto a redisep® R_{f}column with gradient elution (0 to 2%) of methanol in dichloromethane to afford the title compound (0.045 g, 46.5%) as a colourless oil. LCMS m/z=598.06 (M+; 100%).

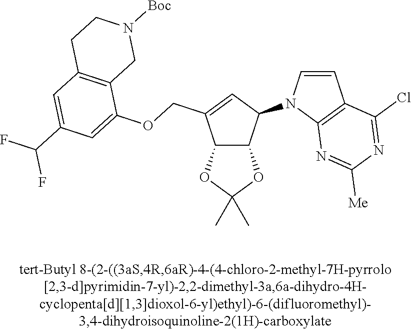 [SMILES: Cc1nc(Cl)c2ccn([C@@H]3C=C(COc4cc(C(F)F)cc5c4CN(C(=O)OC(C)(C)C)CC5)[C@H]4OC(C)(C)O[C@H]43)c2n1]

4-Chloro-7-((3aS,4R,6aR)-2,2-dimethyl-6-vinyl-3a,6a-dihydro-4H-cyclopenta[d][1,3]dioxol-4-yl)-2-methyl-7H-pyrrolo[2,3-d]pyrimidine (0.100 g, 0.301 mmol) in 9-BBN (1.808 ml, 0.904 mmol) was heated at 60° C. for 1 h under N₂ atmosphere. The reaction mixture was cooled to 25° C., then potassium phosphate tribasic (0.192 g, 0.904 mmol) in water (0.2 ml) was added and stirred for 15 mins. A solution of tert-butyl 8-bromo-6-(difluoromethyl)-3,4-dihydroisoquinoline-2(1H)-carboxylate (0.109 g, 0.301 mmol) in THF (1 ml) was added, followed by PdCl₂(dppf)—CH₂Cl₂ adduct (8.61 mg, 10.55 mol). The resulting mixture was stirred at 60° C. for 4 h. The reaction mixture was diluted with ethyl acetate (10 ml) and washed with water (10 ml). Layers were separated, and the organic layer was washed with brine (10 ml) and dried over anhydrous Na₂SO₄. The organic layer was filtered and concentrated in vacuo to give 0.11 g of a crude compound. This residue was purified by combiflash (R_{f}200, Teledyne/Isco) instrument onto a redisep® R_{f} column with gradient elution (0 to 20%) of ethyl acetate in petroleum ether to afford the title compound (0.034 g, 18.34%) as an off-white solid. ¹H NMR (400 MHz, DMSO-d₆) δ 7.34 (s, 1H), 7.23 (d, J=23.4 Hz, 2H), 6.96 (t, J=56.0 Hz, 1H), 6.57 (d, J=3.7 Hz, 1H), 5.69 (s, 1H), 5.59 (d, J=2.4 Hz, 1H), 5.43 (d, J=5.6 Hz, 1H), 4.57 (s, 3H), 3.55 (s, 2H), 2.97-2.78 (m, 4H), 2.66 (s, 3H), 2.60-2.54 (m, 2H), 1.43-1.36 (m, 12H), 1.30 (s, 3H). LCMS m/z=614.95 (M+; 90%).

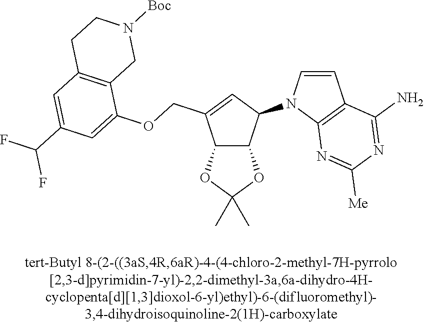 [SMILES: Cc1nc(N)c2ccn([C@@H]3C=C(COc4cc(C(F)F)cc5c4CN(C(=O)OC(C)(C)C)CC5)[C@H]4OC(C)(C)O[C@H]43)c2n1]

To a mixture of tert-butyl 8-(2-((3aS,4R,6aR)-4-(4-chloro-2-methyl-7H-pyrrolo[2,3-d]pyrimidin-7-yl)-2,2-dimethyl-3a,6a-dihydro-4H-cyclopenta[d][1,3]dioxol-6-yl)ethyl)-6-(difluoromethyl)-3,4-dihydroisoquinoline-2(1H)-carboxylate (0.250 g, 0.406 mmol) in dioxane (2 ml) was added aq. ammonia (1.759 ml, 81 mmol) at 25° C. and stirred the reaction mixture at 120° C. for 16 h in a steel bomb. Volatiles were removed in vacuo and the crude residue was purified by combiflash (R_{f}200, Teledyne/Isco) instrument onto a redisep® R_{f}column with gradient elution (0 to 2%) of methanol in dichloromethane to afford the title compound (0.21 g, 87%) as a colourless oil. LCMS m/z=496.3 (M-100; 50%).

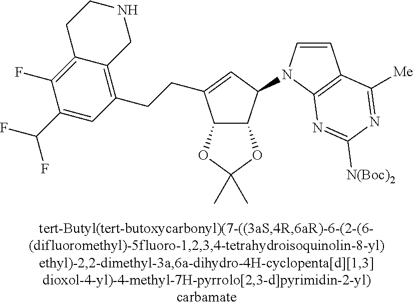

To a stirred solution of tert-butyl (tert-butoxycarbonyl)(7-((3aS,4R,6aR)-6-(2-(6-(difluoromethyl)-5-fluoroisoquinolin-8-yl)ethyl)-2,2-dimethyl-3a,6a-dihydro-4H-cyclopenta[d][1,3]dioxol-4-yl)-4-methyl-7H-pyrrolo[2,3-d]pyrimidin-2-yl)carbamate (0.350 g, 0.493 mmol) in acetic acid (7 ml) was added NaBH₄ (0.065 g, 1.726 mmol) portion-wise at 25° C. and stirred for 2 h. Volatiles were removed in vacuo and the residue dissolved in dichloromethane (15 ml) and basified with saturated aqueous NaHCO₃. Layers were separated, organic layer was washed with brine (10 ml) and dried over anhydrous Na₂SO₄. The organic layer was filtered and concentrated in vacuo to give (0.25 g, 71%) of a crude compound. LCMS m/z=714.52 (M+; 100%).

### Examples

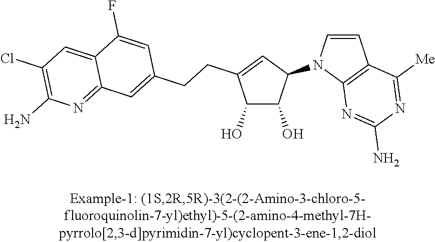 [SMILES: Cc1nc(N)nc2c1ccn2[C@@H]1C=C(CCc2cc(F)c3cc(Cl)c(N)nc3c2)[C@@H](O)[C@H]1O]

A stirred solution of diboc-7-(2-((3aS,4R,6aR)-4-(2-amino-4-methyl-7H-pyrrolo[2,3-d]pyrimidin-7-yl)-2,2-dimethyl-3a,6a-dihydro-4H-cyclopenta[d][1,3]dioxol-6-yl)ethyl)-3-chloro-5-fluoro-N,N-bis(4-methoxybenzyl)quinolin-2-amine (1.10 g, 1.158 mmol) in TFA (8.93 ml, 116 mmol) was stirred at 60° C. for 5 h under N₂ atmosphere. Volatiles were removed in vacuo and the residue was diluted with MeOH (15 ml) followed by addition of K₂CO₃ (0.801 g, 5.79 mmol). The resulting mixture was stirred at 60° C. for 1.5 h under N₂ atmosphere. Volatiles were removed in vacuo and the crude residue was purified by reverse phase preparative HPLC (YMC Triart C18 250×50 mm,10 μm) with gradient elution of mobile phase B in mobile phase A (100% mobile phase A to 50:50 mobile phase A: mobile phase B; Mobile Phase A: Water:CH₃CN (95:5, V/V)+0.1% NH₄OH, Mobile Phase B: CH₃CN:Water (90:10,V/V)+0.1% NH₄OH; Wavelength: 220 nm; Flow Rate: 117 ml/min; Run time: 20 min) to afford the title compound (0.101 g, 18.59%) as an off white solid. ¹H NMR (400 MHz, DMSO-d₆) δ 8.18 (s, 1H), 7.21 (s, 1H), 7.00 (dd, J=11.1, 1.4 Hz, 1H), 6.96 (s, 2H), 6.48 (d, J=3.7 Hz, 1H), 6.20 (d, J=3.6 Hz, 1H), 6.07 (s, 2H), 5.40 (d, J=7.2 Hz, 2H), 4.93 (dd, J=25.6, 6.2 Hz, 2H), 4.45 (t, J=6.0 Hz, 1H), 3.98-3.89 (m, 1H), 3.02-2.85 (m, 2H), 2.57-2.52 (m, 2H), 2.38 (s, 3H).; LCMS m/z=469.23 (M+; 50%).

Examples in table-1 were synthesized by following an analogous reaction protocol as was used for the preparation of (1S,2R,5R)-3-(2-(2-Amino-3-chloro-5-fluoroquinolin-7-yl)ethyl)-5-(2-amino-4-methyl-7H-pyrrolo[2,3-d]pyrimidin-7-yl)cyclopent-3-ene-1,2-diol using the appropriate starting materials.

TABLE 1

| Structure & IUPAC name | Intermediate used | ¹H NMR & LCMS data |
|---|---|---|
| Example-2: 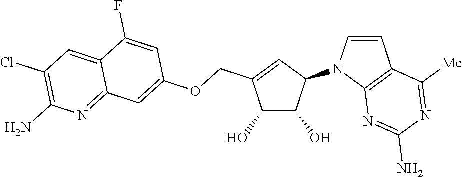 [SMILES: Cc1nc(N)nc2c1ccn2[C@@H]1C=C(COc2cc(F)c3cc(Cl)c(N)nc3c2)[C@@H](O)[C@H]1O] (1S,2R,5R)-3-(((2-Amino-3-chloro-5-fluoroquinolin-7-yl)oxy)methyl)-5- (2-amino-4-methyl-7H-pyrrolo[2,3-d]pyrimidin-7-yl)cyclopent-3-ene-1,2-diol | tert-Butyl (7-((3aS,4R,6aR)- 6-(((2-(bis(4-methoxybenzyl) amino)-3-chloro-5- fluoroquinolin-7- yl)oxy)methyl)-2,2- dimethyl-3a,6a-dihydro-4H- cyclopenta[d][1,3]dioxol- 4-yl)-4-methyl-7H- pyrrolo[2,3-d]pyrimidin- 2-yl)(tert-butoxycarbonyl) carbamate | ¹H NMR (400 MHz, DMSO-d₆) δ 8.09 (s, 1H), 6.93 (s, 2H), 6.88-6.78 (m, 3H), 6.37 (d, J = 3.7 Hz, 1H), 6.08 (s, 2H), 5.76 (d, J = 2.0 Hz, 1H), 5.51 (d, J = 5.0 Hz, 1H), 5.15 (d, J = 6.1 Hz, 1H), 5.09 (d, J = 6.6 Hz, 1H), 4.91- 4.76 (m, 2H), 4.57 (t, J = 5.8 Hz, 1H), 4.17-4.11 (m, 1H), 2.41 (s, 3H). LCMS m/z = 471.11 (M+; 100%). |
| Example-3: 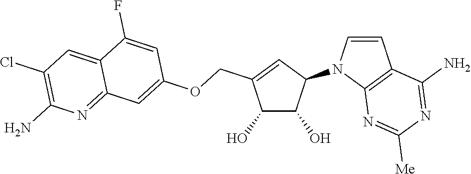 [SMILES: Cc1nc(N)c2ccn([C@@H]3C=C(COc4cc(F)c5cc(Cl)c(N)nc5c4)[C@@H](O)[C@H]3O)c2n1] (1S,2R,5R)-5-(4-Amino-2-methyl-7H-pyrrolo[2,3-d]pyrimidin-7-yl)-3- (((2-amino-3-chloro-5-fluoroquinolin-7-yl)oxy)methyl)cyclopent-3-ene-1,2-diol | tert-Butyl (7-((3aS,4R,6aR)- 6-(((2-(bis(4-methoxybenzyl) amino)-3-chloro-5- fluoroquinolin-7- yl)oxy)methyl)-2,2- dimethyl-3a,6a-dihydro-4H- cyclopenta[d][1,3]dioxol- 4-yl)-2-methyl-7H- pyrrolo[2,3-d]pyrimidin- 4-yl)(tert-butoxycarbonyl) carbamate | ¹H NMR (400 MHz, DMSO-d₆) δ 8.09 (s, 1H), 7.14 (s, 2H), 6.94 (d, J = 3.7 Hz, 3H), 6.86-6.80 (m, 2H), 6.54 (d, J = 3.6 Hz, 1H), 5.81- 5.75 (m, 1H), 5.61 (d, J = 5.2 Hz, 1H), 5.17 (d, J = 6.4 Hz, 2H), 4.92-4.77 (m, 2H), 4.56 (t, J = 5.9Hz, 1H), 4.15 (q, J = 5.6 Hz, 1H), 2.38 (s, 3H).; LCMS m/z = 471.11 (M+; 50%). |
| Example-4: 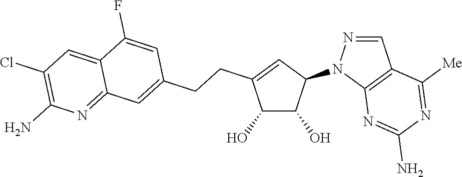 [SMILES: Cc1nc(N)nc2c1cnn2[C@@H]1C=C(CCc2cc(F)c3cc(Cl)c(N)nc3c2)[C@@H](O)[C@H]1O] (1S,2R,5R)-3-(2-(2-Amino-3-chloro-5-fluoroquinolin-7-yl)ethyl)-5-(6- amino-4-methyl-1H-pyrazolo[3,4-d]pyrimidin-1-yl)cyclopent-3-ene-1,2-diol | tert-Butyl (1-((3aS,4R,6aR)-6-(2- (2-(bis(4-methoxybenzyl) amino)-3-chloro-5- fluoroquinolin-7- yl)ethyl)-2,2-dimethyl- 3a,6a-dihydro-4H- cyclopenta[d][1,3]dioxol- 4-yl)-4-methyl-1H-pyrazolo[3,4- d]pyrimidin-6-yl)(tert- butoxycarbonyl)carbamate | ¹H NMR (400 MHz, DMSO-d6) δ 8.16 (s, 1H), 7.99 (s, 1H), 7.21 (s, 1H), 7.05-6.90 (m, 3H), 6.74 (s, 2H), 5.51-5.41 (m, 2H), 5.00- 4.85 (m, 2H), 4.45 (t, J = 5.9 Hz, 1H), 4.35-4.26 (m, 1H), 2.99-2.82 (m, 2H), 2.5-2.53 (m, 5H); LCMS m/z = 469.98 (M+; 10%). |

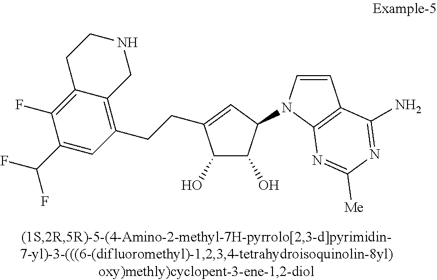 [SMILES: Cc1nc(N)c2ccn([C@@H]3C=C(CCc4cc(C(F)F)c(F)c5c4CNCC5)[C@@H](O)[C@H]3O)c2n1]

To a stirred solution of tert-butyl 8-(((3aS,4R,6aR)-4-(4-amino-2-methyl-7H-pyrrolo[2,3-d]pyrimidin-7-yl)-2,2-dimethyl-3a,6a-dihydro-4H-cyclopenta[d][1,3]dioxol-6-yl)methoxy)-6-(difluoromethyl)-3,4-dihydroisoquinoline-2(1H)-carboxylate (0.190 g, 0.318 mmol) in dioxane (3 ml) was added dioxane HCl (1.590 ml, 6.36 mmol) at 0° C. and stirred the reaction mixture at 25° C. for 3 h. Volatiles were removed in vacuo and basified with methanolic ammonia (7N). Volatiles were removed in vacuo to dryness and the crude residue was purified by reverse phase preparative HPLC (YMC Triart C18 250×50 mm,10 μm) with gradient elution of mobile phase B in mobile phase A (100% mobile phase A to 20:80 mobile phase A: mobile phase B; Mobile Phase A: Water:CH₃CN (95:5, V/V)+0.1% NH₄OH, Mobile Phase B: CH₃CN:Water (90:10,V/V)+0.1% NH₄OH; Wavelength: 220 nm; Flow Rate: 117 ml/min; Run time: 25 min) to afford the title compound (0.021 g, 14.44%) as an off white solid. 1H NMR (400 MHz, DMSO-d6) δ 6.99 (s, 1H), 6.92 (s, 1H), 6.90 (s, 1H), 6.86 (d, J=3.6 Hz, 3H), 6.49 (d, J=3.5 Hz, 1H), 5.73 (d, J=1.9 Hz, 1H), 5.62 (d, J=4.1 Hz, 1H), 5.11 (dd, J=13.4, 6.6 Hz, 2H), 4.78 (s, 2H), 4.56 (t, J=6.0 Hz, 1H), 4.12-4.08 (m, 1H), 3.79 (s, 2H), 2.88 (t, J=5.7 Hz, 2H), 2.70-2.65 (m, 2H), 2.55 (s, 3H); LCMS m/z=458.17 (M+; 20%).

Examples in table-2 were synthesized by following an analogous reaction protocol as was used for the preparation of (1S,2R,5R)-5-(4-amino-2-methyl-7H-pyrrolo[2,3-d]pyrimidin-7-yl)-3-(((6-(difluoromethyl)-1,2,3,4-tetrahydroisoquinolin-8-yl)oxy)methyl)cyclopent-3-ene-1,2-diol using the appropriate starting materials.

| Structure & IUPAC name | Intermediate used | ¹H NMR & LCMS data |
|---|---|---|
| Example-6: 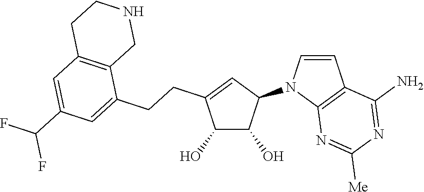 [SMILES: Cc1nc(N)c2ccn([C@@H]3C=C(CCc4cc(C(F)F)cc5c4CNCC5)[C@@H](O)[C@H]3O)c2n1] (1S,2R,5R)-5-(4-Amino-2-methyl-7H-pyrrolo[2,3- d]pyrimidin-7-yl)-3-(2-(6-(difluoromethyl)-1,2,3,4- tetrahydroisoquinolin-8-yl)ethyl)cyclopent-3-ene-1,2-diol | tert-Butyl 8-(2- ((3aS,4R,6aR)-4-(4- amino-2-methyl-7H- pyrrolo[2,3-d]pyrimidin- 7-yl)-2,2-dimethyl-3a,6a- dihydro-4H- cyclopenta[d][1,3]dioxol- 6-yl)ethyl)-6- (difluoromethyl)-3,4- dihydroisoquinoline- 2(1H)-carboxylate | ¹H NMR (400 MHz, DMSO-d6) δ 7.20 (s, 1H), 7.12 (s, 1H), 7.01-6.79 (m, 3H), 6.76 (d, J = 3.5 Hz, 1H), 6.46 (d, J = 3.5 Hz, 1H), 5.53 (d, J = 4.2 Hz, 1H), 5.48 (t, J = 1.7 Hz, 1H), 4.99 (d, J = 6.1 Hz, 1H), 4.95 (d, J = 6.4 Hz, 1H), 4.46 (t, J = 5.8 Hz, 1H), 3.99 (q, J = 5.5 Hz, 1H), 3.90 (s, 2H), 2.91 (t, J = 5.8 Hz, 2H), 2.83- 2.71 (m, 4H), 2.44 (d, J = 9.3 Hz, 2H), 2.36 (s, 3H); LCMS m/z = 456.4 (M + 1; 20%). |

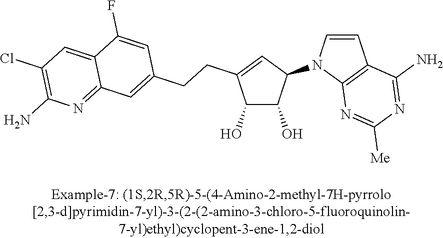 [SMILES: Cc1nc(N)c2ccn([C@@H]3C=C(CCc4cc(F)c5cc(Cl)c(N)nc5c4)[C@@H](O)[C@H]3O)c2n1]

A mixture of 7-(2-((3aS,4R,6aR)-4-(4-amino-2-methyl-7H-pyrrolo[2,3-d]pyrimidin-7-yl)-2,2-dimethyl-3a,6a-dihydro-4H-cyclopenta[d][1,3]dioxol-6-yl)ethyl)-3-chloro-5-fluoroquinolin-2-amine (0.040 g, 0.079 mmol) and TFA (0.605 ml, 7.86 mmol) was stirred at 25° C. for 1 h. Volatiles were removed in vacuo and basified with 7N methanolic ammonia. Again volatiles were removed in vacuo to dryness and the crude residue was purified by combiflash (R_{f}200, Teledyne/Isco) instrument onto a redisep® R_{f}column with gradient elution (0 to 7%) of methanol in dichloromethane to afford the title compound (0.033 g, 90%) as an off white solid. ¹H NMR (400 MHz, DMSO-d6) δ 8.17 (s, 1H), 7.21 (s, 1H), 7.01 (dd, J=11.0, 1.4 Hz, 1H), 6.96 (s, 2H), 6.84 (s, 2H), 6.54 (d, J=3.5 Hz, 1H), 6.36 (d, J=3.5 Hz, 1H), 5.54-5.47 (m, 1H), 5.43-5.38 (m, 1H), 4.98 (d, J=6.3 Hz, 1H), 4.94 (d, J=6.6 Hz, 1H), 4.45 (t, J=6.1 Hz, 1H), 3.97-3.89 (m, 1H), 2.99-2.89 (m, 2H), 2.60-2.54 (m, 2H), 2.35 (s, 3H).; LCMS m/z=469.17 (M+; 20%).

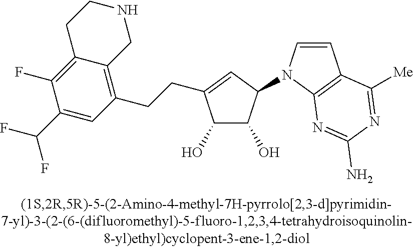 [SMILES: Cc1nc(N)nc2c1ccn2[C@@H]1C=C(CCc2cc(C(F)F)c(F)c3c2CNCC3)[C@@H](O)[C@H]1O]

A mixture of tert-butyl (tert-butoxycarbonyl)(7-((3aS,4R,6aR)-6-(2-(6-(difluoromethyl)-5-fluoro-1,2,3,4-tetrahydroisoquinolin-8-yl)ethyl)-2,2-dimethyl-3a,6a-dihydro-4H-cyclopenta[d][1,3]dioxol-4-yl)-4-methyl-7H-pyrrolo[2,3-d]pyrimidin-2-yl)carbamate (0.250 g, 0.350 mmol) and TFA (1.349 ml, 17.51 mmol) was stirred at room temperature for 15 h. Volatiles were removed in vacuo and basified with 7N methanolic ammonia. This crude residue was purified by reverse phase preparative HPLC (YMC Triart C18 250×50 mm,10 μm) with gradient elution of mobile phase B in mobile phase A (100% mobile phase A to 20:80 mobile phase A: mobile phase B; Mobile Phase A: Water:CH₃CN (95:5, V/V)+0.1% NH₄OH, Mobile Phase B: CH₃CN:Water (90:10,V/V)+0.1% NH₄OH; Wavelength: 220 nm; Flow Rate: 117 ml/min; Run time: 24 min) to afford the title compound (0.047 g, 28.3%) as a pale yellow solid. ¹H NMR (400 MHz, DMSO-d6) δ 7.33-6.99 (m, 2H), 6.72 (d, J=3.7 Hz, 1H), 6.36 (d, J=3.6 Hz, 1H), 6.08 (s, 2H), 5.50-5.42 (m, 2H), 4.95 (dd, J=15.8, 6.2 Hz, 2H), 4.47 (t, J=5.8 Hz, 1H), 3.99 (s, 3H), 3.17 (d, J=3.5 Hz, 1H), 3.05-3.00 (m, 1H), 2.87-2.63 (m, 5H), 2.56 (s, 1H), 2.41 (s, 3H). LCMS m/z=474.36 (M+; 20%).

### Biological Examples

### Biochemical Assay Protocol 1

Inhibitory effect of compounds on PRMT5 was assessed using HTRF detection technology in biochemical assay. Biotinylated H4R3 (residues 1-21) was used as a substrate. Compounds were pre-incubated with 15-25 ng PRMT5:MEP50 per well of a 384-well plate for 30 min at room temperature in the assay buffer containing 20 mM Bicine, pH 7.6, 25 mM NaCl, 2 mM DTT, 0.01% Chicken albumin and 0.01% Tween-20. Reaction was initiated by adding 1 μM of SAM and 50 nM biotinylated H4R3. Total assay volume was 15 μL. Reaction was continued for 120 min at room temperature. Then detection solution containing Streptavidin-Eu cryptate, anti-rabbit IgG-XL-665, Histone H4R3 Dimethyl Symmetric (H4R3me2s) Polyclonal Antibody, all prepared in HTRF detection buffer was added and further incubated for 30 min at room temperature. HTRF signal was recorded in PHERAStar microplate reader. Ratio of signal obtained at 665 nm and 620 nm was used to compute the percent inhibition of compound as follows:

% Inhibition=100−((Test Ratio−Negative control Ratio)/(Positive control Ratio−Negative control Ratio)*100) where
Positive control=PRMT5+SAM+H4R3
Negative control=PRMT5+H4R3

### Biochemical Assay Protocol 2

Inhibitory effect of compounds on PRMT5 was assessed using HTRF detection technology in biochemical assay. Biotinylated H4R3 (residues 1-21) was used as a substrate. Compounds were pre-incubated with 2.5 ng PRMT5:MEP50 per well of a 384-well plate for 30 min at room temperature in the assay buffer containing 20 mM Bicine, pH 7.6, 25 mM NaCl, 2 mM DTT, 0.01% Chicken albumin and 0.01% Tween-20. Reaction was initiated by adding 1 μM of SAM and 50 nM biotinylated H4R3. Total assay volume was 15 μL. Reaction was continued for 4 h at room temperature. Then detection solution containing Streptavidin-Eu cryptate, anti-rabbit IgG-XL-665, Histone H4R3 Dimethyl Symmetric (H4R3me2s) Polyclonal Antibody, all prepared in HTRF detection buffer was added and further incubated for 30 min at room temperature. HTRF signal was recorded in PHERAStar microplate reader. Ratio of signal obtained at 665 nm and 620 nm was used to compute the percent inhibition of compound as follows

% Inhibition=100−((Test Ratio−Negative control Ratio)/(Positive control Ratio−Negative control Ratio)*100) where
Positive control=PRMT5+SAM+H4R3
Negative control=PRMT5+H4R3

| Activity Range | Example # |
|---|---|
| IC₅₀ 400 pM to 750 pM | 1, 2, 3, 4, 7, 8 |
| IC₅₀ > 750 pM | 5, 6 |

### SDMA Inhibition Assay

### Protocol

Z-138 cells (ATCC, CRL-3001™) were seeded at a density of 1 million cells/well in transparent, flat bottomed tissue culture grade 48-well plates. Cells were treated with various concentration of test compounds for a period of 48 h. Cell lysate was prepared using 1×CST Lysis buffer (Cell Signaling Technology, USA) and 500 ng/well/50 μL of lysate in pH 9.6 carbonate buffer was coated on 96-well Maxisorb plate and incubated overnight at 4° C. The plate was washed twice in 1×PBS containing 0.05% Tween 20 and blocked in 1% BSA for 1 h at ambient temperature. Further, the plate was incubated first with primary antibody (anti-SDMA antibody; CST #13222s) at ambient temperature for 2 h and then with HRP-conjugated secondary antibody at ambient temperature for 1 h with 2 intermittent washing steps in between.

For luminescence-based detection, HRP substrates (substrate A+substrate B in a 1:1 proportion) were added followed by luminescence reading after 30 min in Synergy™ 2 reader (Biotek, USA).

For absorbance-based detection, TMB substrate was added followed by addition of STOP solution (2N H₂SO₄) post colour development and absorbance (excitation 450 nm and emission 540 nm) was measured in Synergy™ 2 reader (Biotek, USA).

% inhibition of SDMA was calculated relative to the vehicle control samples containing media with 0.1% DMSO alone as per the formula below.

$$\frac{( Avg . of Untreated Control - Avg . of Test )}{Avg . of Untreated control} \times 100$$

The IC₅₀ values of individual compounds were calculated with Non Linear Regression Analysis using Graph Pad Prism (Graph Pad software, Inc, USA).

| Activity Range | Example # |
|---|---|
| IC₅₀ 100 pM to 1 nM | 1, 4, 7 |
| IC₅₀ 1.1 nM to 50 nM | 2, 3, 8 |

### Anticancer Activity Assay

Z-138 cells were seeded at a density of 2000-3000 cells per well in culture media (IMDM+10% FBS). PANC-1 (ATCC, CRL-1469™) and MIA PaCa-2 (ATCC, CRL-1420™) cells were seeded at a density of 200-300 cells per well in culture media (DMEM+10% FBS). Cells were seeded in opaque, flat bottomed tissue culture grade 96-well plates and Z-138 cells (suspension) were seeded and treated on the same day with various concentrations of test compounds. PANC-1 and MIA PaCa-2 cells, being adherent, were kept for overnight settlement at standard cell culture conditions (37° C., 5% CO₂). On the following day, cells were treated with various concentrations of test compounds. Cells were treated with test compounds for a period of 96 h, 7 days and 10 days, for Z-138 cells, PANC-1 cells and MIA PaCa-2 cells, respectively. Cell viability was assessed using CellTiterGlo™ (Promega, USA) as per manufacturer's instructions. Relative Light Units (RLU) were read in Synergy™ 2 reader (Biotek, USA). The assay measures cellular ATP as an indicator of cell viability. RLU is proportional to the number of viable cells in the respective well.

% inhibition of cell viability was calculated relative to the vehicle control samples containing media with 0.1% DMSO alone as per the formula below.

$$\frac{( Avg . of Untreated Control - Avg . of Test )}{Avg . of Untreated control} \times 100$$

The IC₅₀ values of individual compounds were calculated with Non Linear Regression Analysis using Graph Pad Prism (Graph Pad software, Inc, USA).

| Activity Range | Example # |
|---|---|
| IC₅₀ 1 pM to 1 nM | 1, 2, 3, 4, 7 |
| IC₅₀ > 1 nM | 5, 6, 8 |

### Anti-Cancer Assay (MIA PaCa-2)

| Activity Range | Example # |
|---|---|
| IC₅₀ 1 pM to 400 pM | 1, 7 |
| IC₅₀ 401 pM to 30 nM | 2, 3, 4 |

### In Vivo Efficacy Experiments

Tumor xenograft for mantle cell lymphoma was established by injection of cells into the right flank of female NOD.CB17-Prkdc<scid>/J mice with an age between 7-11 weeks purchased from The Jackson Laboratory, USA. All animal study proposals were reviewed and approved by the Institutional Animal Ethics Committee (IAEC) prior to initiation of experimentation.

### Z-138 Xenograft

For Z-138 xenograft mouse model, Z-138 cells (ATCC® CRL-3001™) were grown in IMDM medium supplemented with 10% FBS. Cells were incubated under standard conditions at 37° C. and 5% CO₂. For generating tumors, Z-138 cells in IMDM medium were mixed with Matrigel (Corning® Matrigel® Basement Membrane Matrix) in a ratio of 1:1. 10×10⁶ cells) in a volume of 200 μL were injected subcutaneously in each mouse to establish tumors. Mice were randomized into treatment groups of 8-10 mice, once tumors reached an average volume between 100 to 120 mm³. Treatment was initiated on day of randomization and continued until end of the study. The Vehicle and test compound treatment groups were administered respective treatments orally, using gavage tubing, at an application volume of 10 mL/kg per mouse twice a day.

Mice were housed in individually ventilated cages (IVC) at room temperature of 22+3° C., humidity 50+20% and 12/12 h light/dark cycle. All the experimental activities were carried-out inside the biosafety cabinets to ensure sterility.

Tumor size was measured with Digimatic Vernier caliper (Mitutoyo, Japan) when the tumors became palpable. Tumor volume (T. V.) is calculated by using the formula:
Tumor volume (mm3)=(L xW2)/2
Where, L: Length of tumor, W: Width of tumor in millimeter
Percent tumor growth inhibition (% TGI) is calculated using the formula:

% TGI=[1−(Tf−Ti)/(Cf−Ci)]×100
Where, Tf and Ti, are the final and initial tumor volumes (test compound), and Cf and Ci are the final and initial mean tumor volumes (vehicle group), respectively.

### Percent Tumor Regression is Calculated as:

% TR:(Ti−Tf)/(Ti)×100
Where, Tf and Ti, are the final and initial tumor volumes, respectively.

### MIA PaCa-2 Xenograft

Tumor fragment xenograft for pancreatic cancer cell line MIA PaCa-2 was established by implantation of tumor fragments 30-45 mm³ subcutaneously into the right flank of female athymic nude FOXn1<nu>/J/Mus musculus mice with an age between 10-11 weeks purchased from the Jackson Laboratory, USA. All animal study proposals were reviewed and approved by the Institutional Animal Ethics Committee (IAEC) prior to initiation of experimentation.

The example #1 was tested for tumor growth inhibition in subchronic MIA PaCa-2 xenograft model using assay procedure given above; the % of tumor growth inhibition at 2 mg/kg dose was found to be 57%.

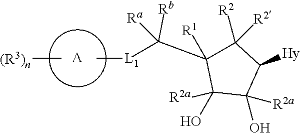

1. A compound of formula (III), its stereoisomer, or its pharmaceutically acceptable salt, 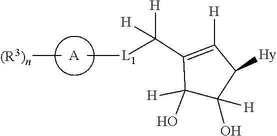
wherein,
L₁ is —CH₂— or O;
ring A is 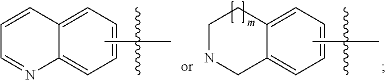
Hy is selected from formula (a-1), (b-1) and (d-1), 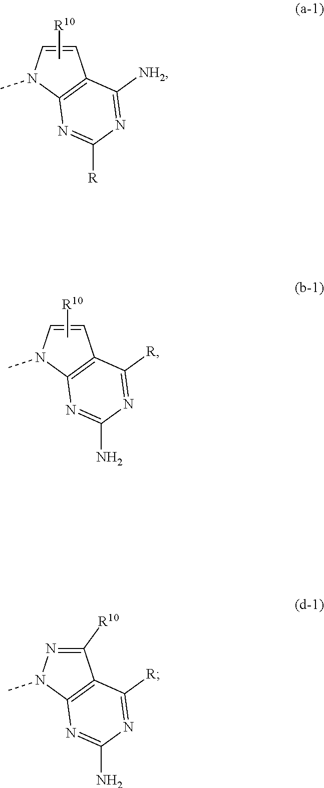
R is methyl; R³ is independently selected at each occurrence from halogen, substituted or unsubstituted alkyl and —NR⁷R⁸; R⁷ and R⁸ are independently selected from hydrogen;
R¹⁰ is hydrogen; ‘m’ is 1; ‘n’ is an integer ranging from 0 to 3, both inclusive; with a proviso that the compound is not (1S,2R,5R)-3-(2-(2-Amino-3-chloro-5-fluoroquinolin-7-yl)ethyl)-5-(2-amino-4-methyl-7H-pyrrolo[2,3-d]pyrimidin-7-yl)cyclopent-3-ene-1,2-diol.

2. The compound of formula (III), its stereoisomer, or its pharmaceutically acceptable salt, as claimed in claim 1, wherein the compound is selected from:
(1S,2R,5R)-3-(((2-Amino-3-chloro-5-fluoroquinolin-7-yl)oxy)methyl)-5-(2-amino-4-methyl-7H-pyrrolo[2,3-d]pyrimidin-7-yl)cyclopent-3-ene-1,2-diol;
(1S,2R,5R)-5-(4-Amino-2-methyl-7H-pyrrolo[2,3-d]pyrimidin-7-yl)-3-(((2-amino-3-chloro-5-fluoroquinolin-7-yl)oxy)methyl)cyclopent-3-ene-1,2-diol;
(1S,2R,5R)-3-(2-(2-Amino-3-chloro-5-fluoroquinolin-7-yl)ethyl)-5-(6-amino-4-methyl-1H-pyrazolo[3,4-d]pyrimidin-1-yl)cyclopent-3-ene-1,2-diol;
(1S,2R,5R)-5-(4-Amino-2-methyl-7H-pyrrolo[2,3-d]pyrimidin-7-yl)-3-(((6-(difluoromethyl)-1,2,3,4-tetrahydroisoquinolin-8-yl)oxy)methyl)cyclopent-3-ene-1,2-diol;
(1S,2R,5R)-5-(4-Amino-2-methyl-7H-pyrrolo[2,3-d]pyrimidin-7-yl)-3-(2-(6-(difluoromethyl)-1,2,3,4-tetrahydroisoquinolin-8-yl)ethyl)cyclopent-3-ene-1,2-diol;
(1S,2R,5R)-5-(4-Amino-2-methyl-7H-pyrrolo[2,3-d]pyrimidin-7-yl)-3-(2-(2-amino-3-chloro-5-fluoroquinolin-7-yl)ethyl)cyclopent-3-ene-1,2-diol, and
(1S,2R,5R)-5-(2-Amino-4-methyl-7H-pyrrolo[2,3-d]pyrimidin-7-yl)-3-(2-(6-(difluoromethyl)-5-fluoro-1,2,3,4-tetrahydroiso quinolin-8-yl)ethyl)cyclopent-3-ene-1,2-diol.

3. A pharmaceutical composition comprising at least one compound of claim 1, its stereoisomer, or its pharmaceutically acceptable salt, and a pharmaceutically acceptable carrier.

4. A method for treating a disease, disorder, syndrome or condition associated with PRMT5 enzyme, comprising administering to a subject in need thereof an effective amount of compound as claimed in claim 1.

5. The method as claimed in claim 4, wherein the said disease, disorder, syndrome, or condition associated with PRMT5 enzyme is glioblastoma multiforme, prostate cancer, pancreatic cancer, mantle cell lymphoma, non-Hodgkin's lymphomas and diffuse large B-cell lymphoma, acute myeloid leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, and cervical cancer.

6. The method as claimed in claim 4, wherein the said disease, disorder, syndrome, or condition associated with PRMT5 enzyme is cancer.

7. Use of a compound of claim 1, or pharmaceutically acceptable salt thereof in the preparation of medicament for treating a disease, disorder, syndrome or condition associated with PRMT5.

8. The use as claimed in claim 7, wherein the disease, disorder, syndrome, or condition associated with PRMT5 are selected from the group consisting of glioblastoma multiforme, prostate cancer, pancreatic cancer, mantle cell lymphoma, non-Hodgkin's lymphomas and diffuse large B-cell lymphoma, acute myeloid leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, and cervical cancer.

9. The use as claimed in claim 7, wherein the disease, disorder, syndrome, or condition associated with PRMT5 is cancer.
